(19) United States
(12) Patent Application Publication
(10) Pub. No.: US 2013/0065885 A1
(43) Pub. Date: Mar. 14, 2013

(54) ORAL THERAPEUTIC COMPOUND DELIVERY SYSTEM

(71) Applicants: Michael Stephen Roberts, West Lake (AU); Ruoying Jiang, Sherwood (AU); Keivan Bezanehtak, Rosebery (AU); Greg Andrew Davey, Sinnamon Park (AU); George Alexander Davidson, Larnook (AU); Geraldine Ann Elliott, Mount Ommaney (AU); Stephen Douglas Chandler, Mayfield (AU); Mantu Sarkar, Fairfield (AU)

(72) Inventors: Michael Stephen Roberts, West Lake (AU); Ruoying Jiang, Sherwood (AU); Keivan Bezanehtak, Rosebery (AU); Greg Andrew Davey, Sinnamon Park (AU); George Alexander Davidson, Larnook (AU); Geraldine Ann Elliott, Mount Ommaney (AU); Stephen Douglas Chandler, Mayfield (AU); Mantu Sarkar, Fairfield (AU)

(73) Assignee: IMAGINOT PTY LTD., Fairfield Gardens (AU)

(21) Appl. No.: 13/618,728

(22) Filed: Sep. 14, 2012

Related U.S. Application Data
(62) Division of application No. 11/597,341, filed on Jun. 11, 2008.
(60) Provisional application No. 60/575,461, filed on May 28, 2004.

(51) Int. Cl. A61K 47/04; A61K 47/32; A61K 31/445; A61K 31/137; A61K 31/404; A61K 31/4196; A61K 31/166; A61K 31/451; A61K 31/485; A61K 31/135; A61K 31/5513; A61K 31/5517; A61K 31/519; A61K 31/4178; A61K 31/422; A61K 31/437; A61K 31/495; A61P 29/00; A61P 25/06; A61P 1/08; A61P 25/20; A61P 11/02; A61P 37/08; A61P 15/00; A61K 47/38

(52) U.S. Cl. 514/220; 514/769; 514/317; 514/653; 514/414; 514/383; 514/619; 514/327; 514/282; 514/646; 514/221; 514/252.16; 514/397; 514/376; 514/300; 514/255.04

(57) ABSTRACT

The present invention relates generally to therapeutic formulations. More particularly, this present invention provides an oral delivery system for a therapeutic compound that is a base, a salt of a base or an amphoteric compound or a salt of a amphoteric compound with pharmacological, physiological or biochemical activity or a proactive form thereof. The present invention even more particularly provides a swallow formulation comprising a therapeutic compound that is a base, a salt of a base, an amphoteric compound or a salt of an amphoteric compound which facilitates the rapid delivery of the therapeutic compound to the circulatory system.

### CROSS-REFERENCE TO RELATED APPLICATIONS

The present application is a divisional application of U.S. patent application Ser. No. 11/597,341 filed on Jun. 11, 2008, which is a national phase of International Patent Application No. PCT/AU05/00759 filed on May 27, 2005, which claims priority under 35 U.S.C. §119(e) from U.S. provisional application No. 60/575,461, filed on May 28, 2004, the content of all of which are hereby incorporated by reference in their entireties.

### FIELD OF THE INVENTION

The invention relates generally to therapeutic formulations. More particularly, the present invention provides an oral delivery system for a therapeutic compound that is a base, a salt of a base, and amphoteric compound or a salt of an amphoteric compound with a pharmacological, physiological or biochemical activity or a proactive form thereof. The present invention even more particularly provides a swallow formulation comprising a therapeutic compound that is a base, a salt of a base, an amphoteric compound or a salt of an amphoteric compound which has exceptionally fast dissolution and thus facilitates the rapid delivery of the therapeutic compound to the circulatory system.

### DESCRIPTION OF THE PRIOR ART

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Improving the rate and extent of absorption of oral formulations of compounds has been the subject of substantial research. In general, once a solid swallow composition reaches the stomach, it undergoes disintegration and/or dissolution and passes into the small intestine where the active ingredient is absorbed across intestinal walls into the circulatory system via the portal vein and liver before reaching the site of action. For some drug absorption is not rate limited, and in this case fast disintegration and fast dissolution of the active ingredient should promote fast absorption in vivo.

Solid dosage forms for oral administration can be categorized into three major groups. Those described as swallow formulations are intended to be swallowed whole. Those described as orally disintegrating or orally dissolving or chewable, are intended to be dispersed or dissolved in the mouth before swallowing. The third group is generally called dispersible or soluble formulations that are intended to be dissolved or dispersed in liquid before administration, such that the patient swallows the resultant solution or dispersion.

Of the group of swallow formulations, some are designed for sustained or delayed release through the use of coatings or other devices that control the site of release of the drug within the gastrointestinal tract. Examples include enteric coated tablets to avoid the local gastric toxicity which occurs with some acidic drugs such as the non-steroidal anti-inflammatory drugs (NSAIDs), and controlled or multiphase release of drugs to allow once daily dosage.

Other swallow formulations may be designed for fast dissolution of the active ingredient, with the aim of achieving fast absorption and fast onset of action. This present invention relates to formulations manufactured as solid dosage forms intended to be swallowed intact, which will achieve fast dissolution and fast absorption of the active ingredient.

The use of sodium bicarbonate and other alkali metal carbonates has been described for a number of different purposes in pharmaceutical dosage forms.

The use of sodium bicarbonate and other alkali metal carbonates, as the base component of an effervescent couple in dosage forms intended for dissolution or dispersion in water prior to administration, is widely recognised. Typically the resultant effervescent solutions or dispersions exhibit fast absorption of the drug contained therein.

The purpose of the present invention is to incorporate the advantages of improved absorption and reproducibility of dispersible and/or soluble formulations into swallow formulations that are more convenient, and remain the preferred dosage form for many patients, particularly for regular use.

Effervescent tablet formulations which are disintegrated and/or dissolved in water prior to administration are well known. Such formulations generally contain effervescent couples such as citric acid and sodium bicarbonate in large amounts. For example, U.S. Pat. No. 6,245,353 describes a tablet containing cetirizine and an effervescent couple for disintegration in water prior to administration. A variety of effervescent formulations which are intended to be dispersed and/or dissolved prior to administration are disclosed for example in U.S. Pat. No. 4,704,269, U.S. Pat. No. 4,309,408 and U.S. Pat. No. 4,942,039.

Some publications teach the inclusion of about 630 mg sodium bicarbonate in swallow tablets so as to provide isotonic conditions in the stomach. U.S. Pat. No. 6,316,025, for example describes a swallow tablet of paracetamol containing 300 mg to 1000 mg of sodium bicarbonate per tablet and a paracetamol to sodium bicarbonate ratio of between 0.74 and 1. Grattan et al., Eur. J. Pharm. Biopharm 49(3): 225-229, 2000, subsequently reported that a formulation with 630 mg sodium bicarbonate provided improved pharmacokinetic outcomes. It was suggested that this was due to an osmotic effect of sodium bicarbonate, which would be isotonic when ingested with 100 mL of water. US Patent Application No. 20040204475 describes a formulation containing sodium bicarbonate and eletriptan. The sodium bicarbonate is administered in an amount to obtain a duodenal concentration approximately isotonic with serum (150 millimoles). The formulations exemplified all contained 630 mg sodium bicarbonate.

U.S. Pat. No. 6,699,885 relates to formulations including omeprazole and bicarbonate or carbonate to protect the omeprazole from gastric acid degradation in amounts from about 250 mg to 4,000 mg, thus comprising the major proportion of the tablet by weight.

US Patent Application No. 20050032867 describes a fast disintegrating and dispersing sumatripan formulation comprising about 5 to about 50% by weight base component. The base component of the formulation reacts with the acid component of the stomach, sumatriptan or acid component of the tablet to generate gas so as to facilitate the disintegration and dispersion of the tablet.

It is widely accepted that raising the pH will inhibit the dissolution of basic compounds. WO 2004/017976 describes a fast dissolving and taste masked oral dosage from comprising the basic compound sildenafil. The specification describes the use of any pharmaceutically acceptable pH raising agent to inhibit dissolution of sildenafil, precenting dissolution of sildenafil in the mouth and thus masking the taste of the sildenafil. Agents that raise or increase the pH include sodium carbonate, sodium bicarbonate, calcium carbonate and magnesium carbonate.

Furthermore, precipitation of the basic compound ondansetron in alkaline solutions containing sodium bicarbonate has been reported (Jaronsinski P F and Hirschfield S, N. Eng. J. Med. 325: 1315-1316, 1001).

A relatively large amount of prior art deals with the use of sodium bicarbonate and other pH modulating agents to affect the absorption of acidic drugs, particularly acidic Non-steroidal Anti-Inflammatory Drugs (NSAIDs) and their salts. WO9744023 deals with the use of sodium and potassium bicarbonate to enhance absorption of salts and diclofenac. U.S. Pat. No. 4,834,966 and others deals with the use of arginine, ibuprofen and sodium bicarbonate formulations to enhance absorption of ibuprofen. U.S. Pat. No. 4,704,405 deals with the use of sodium sulindac, a base and a bicarbonate to improve absorption of sulindac. The enhanced absorption results from the increased solubility of acidic drugs at elevated pH, owing to greater ionisation of acidic groups. Neuvonen, P. J and Kivisto, K. T. (Clin. Pharmacokinet. 27 (2) 120-8, 1994.) state that several drugs show enhanced absorption in the presence of pH modulating agents such as common antacids of sodium bicarbonate and magnesium hydroxide due to these antacids increasing gastric pH and thus increasing solubility.

Dissolution testing provides an in vitro method to predict the absorption of formulations based on in vitro in vivo correlations (IVIVC) which have been well documented (Amidon G L et al, Pharm Res, 1995, 12 (3) 413-20, Balan G, et al, Journal of Pharmaceutical Sciences, 2001, 90 (8) 1176-1185, Rostami-Hodjegan A et al, Drug Dev Ind Pharm, 2002, 28 (5) 533-43). Different dissolution media can be used to simulate drug dissolution in the acidic environment of the stomach and the alkaline environment of the small intestine whence absorption occurs. They can also be used to assess the effect of different formulations in modifying gastric pH and the impact of this on drug dissolution. For fast absorption in vivo, the ideal formulation will release drug quickly into solution in the acidic conditions of the stomach before it transfers to the more alkaline conditions in the small intestine.

Once a swallow formulation has disintegrated, other factors such as the intrinsic solubility and surface area of the drug will determine its rate of dissolution under different pH conditions. The dissolution can be further enhanced if the particle size of the drug is reduced to increase the surface area of the drug available for dissolution. However fast disintegration is not always associated with fast dissolution.

On the basis of these disclosures, it would be expected that the addition of bases such as carbonates to therapeutic compounds that are bases, salts of bases, amphoteric compounds or salts of amphoteric compounds, will reduce their solubility and hence dissolution as a result of the increased pH. Unexpectedly, we have found that for swallow formulations in the case of basic and amphoteric drugs, where increased pH is likely to lead to lower solubility and hence worse dissolution and absorption, the use of pH modulating agents can still achieve increased dissolution and potentially increased absorption. Furthermore if a carbonate is used in a swallow formulation, with the level optimized for each drug, then enhanced dissolution can always be achieved, particularly for drugs with limited solubility.

In accordance with the present invention, therapeutic compositions are defined in which the addition of bases such as carbonates, to therapeutic compounds that are bases, salts of bases, amphoteric compounds or salts amphoteric compounds, enable enhances in vitro dissolution of the therapeutic agent.

### BRIEF DESCRIPTION OF THE DRAWINGS

FIG. 1 shows the fexofenadine hydrochloride dissolution profiles in 900 mL 0.0033 N hydrochloric acid at 30 rpm for formulation 1, formulation 2 and the commercial product.

FIG. 2 shows the fexofenadine hydrochloride dissolution profiles in 900 mL 0.0033 N hydrochloric acid at 0 rpm for formulation 1, formulation 2 and the commercial product.

FIG. 3 shows the pseudoephedrine hydrochloride dissolution profiles in 900 mL 0.0033 N hydrochloric acid at 30 rpm for formulation 1, formulation 2 and the commercial product.

FIG. 4 shows the pseudoephedrine hydrochloride dissolution profiles in 900 mL 0.0033 N hydrochloric acid at 0 rpm for formulation 1 and formulation 2.

FIG. 5 shows the eletriptan hydrobromide dissolution profiles in 900 mL 0.0033 N hydrochloric acid at 30 rpm for formulation 1, formulation 2 and the commercial product.

FIG. 6 shows the eletriptan hydrobromide dissolution profiles in 900 mL 0.0033 N hydrochloric acid at 0 rpm for formulation 2 and the commercial product.

FIG. 7 shows the rizatriptan benzoate dissolution profiles in 900 mL 0.0033 N hydrochloric acid at 30 rpm for formulation 1, formulation 2 and the commercial product.

FIG. 8 shows the rizatriptan benzoate dissolution profiles in 900 mL 0.0033 N hydrochloric acid at 0 rpm for formulation 2.

FIG. 9 shows the metoclopramide hydrochloride dissolution profiles in 900 mL 0.0033 N hydrochloric acid at 30 rpm for formulation 1, formulation 2 and the commercial product.

FIG. 10 shows the metoclopramide hydrochloride dissolution profiles in 900 mL 0.0033 N hydrochloric acid at 0 rpm for formulation 1 and formulation 2.

FIG. 11 shows the loperamide hydrochloride dissolution profiles in 900 mL 0.0033 N hydrochloric acid at 30 rpm for formulation 1, formulation 2 and the commercial product.

FIG. 12 shows the loperamide hydrochloride dissolution profiles in 900 mL 0.0033 N hydrochloric acid at 0 rpm for formulation 1 and formulation 2.

FIG. 13 shows the codeine phosphate dissolution profiles in 900 mL 0.0033 N hydrochloric acid at 30 rpm for formulation 1, formulation 2 and the commercial product.

FIG. 14 shows the codeine phosphate dissolution profiles in 900 mL 0.0033 N hydrochloric acid at 0 rpm for formulation 1 and formulation 2.

FIG. 15 shows the tramadol hydrochloride dissolution profiles in 900 mL 0.0033 N hydrochloric acid at 30 rpm for formulation 2 and the commercial product.

FIG. 16 shows the tramadol hydrochloride dissolution profiles in 900 mL 0.0033 N hydrochloric acid at 0 rpm for formulation 1 and the commercial product.

FIG. 17 shows the diazepam dissolution profiles in 900 mL 0.0033 N hydrochloric acid at 30 rpm for formulation 1, formulation 2 and the commercial product.

FIG. 18 shows the diazepam dissolution profiles in 900 mL 0.0033 N hydrochloric acid at 0 rpm for formulation 1 and formulation 2.

FIG. 19 shows the lorazepam dissolution profiles in 900 mL 0.0033 N hydrochloric acid at 30 rpm for formulation 1, formulation 2 and the commercial product.

FIG. 20 shows the lorazepam dissolution profiles in 900 mL 0.0033 N hydrochloric acid at 0 rpm for formulation 1 and formulation 2.

FIG. 21 shows the alprazolam dissolution profiles in 900 mL 0.0033 N hydrochloric acid at 30 rpm for formulation 1, formulation 2 and the commercial product.

FIG. 22 shows the alprazolam dissolution profiles in 900 mL 0.0033 N hydrochloric acid at 0 rpm formulation 1 and formulation 2.

FIG. 23 shows the dissolution profiles in 900 mL 0.0033 N hydrochloric acid at 30 rpm for the B1 (base alone) and B2 (base+acid) formulations of paracetamol and tramadol hydrochloride, and for the commercial products.

FIG. 24 shows the dissolution Profiles in 900 mL 0.0033 N hydrochloric acid at 0 rpm for the B1 (base alone) and B2 (base+acid) formulations of paracetamol and tramadol hydrochloride.

### SUMMARY OF THE INVENTION

The present invention relates generally to therapeutic formulations and more particularly fast dissolving swallow formulations for a therapeutic compound that is a base, a salt of a base, an amphoteric compound or a salt of an amphoteric compound with pharmacological, physiological or biochemical activity or a proactive form thereof.

The present invention provides a swallow formulation comprising
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralise 0.01 to 9.0 millimoles of hydrochloric acid and is present in an amount from about 1% to 50% by weight of the swallow formulation,
wherein at least about 70% of the therapeutic compound is dissolved from the swallow formulation within 180 seconds, at 30 rpm when the dissolution is measured in United States Pharmacopoeia (USP) dissolution apparatus 2 with 900 mL 0.0033 N hydrochloric acid at 37° C.

In one embodiment of the invention at least about 90% of the therapeutic compound is dissolved from the swallow formulation within 180 seconds at 30 rpm in USP dissolution apparatus 2 with 900 mL 0.0033 N hydrochloric acid at 30 rpm and 37° C.

The present invention further provides a swallow formulation comprising
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralise 0.01 to 9.0 millimoles of hydrochloric acid and is present in an amount from about 1% to 50% by weight of the swallow formulation,
wherein at least about 5% of the therapeutic compound is dissolved from the swallow formulation within 300 seconds at 0 rpm when the dissolution is measured in United States Pharmacopoeia (USP) dissolution apparatus 2 with 900 mL 0.0033 N hydrochloric acid at 37° C.

In one embodiment of the invention at least about 20% or the therapeutic compound is dissolved from the swallow formulation within 300 seconds at 30 rpm in USP dissolution apparatus 2 with 900 mL 0.0033 N hydrochloric acid at 30 rpm and 37° C.

The present invention further provides a swallow formulation comprising
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralise 0.01 to 9.0 millimoles of hydrochloric acid and is present in an amount from about 1% to 50% by weight of the swallow formulation,
wherein
(i) at least about 70% of the therapeutic compound is dissolved from the swallow formulation within 180 seconds, at 30 rpm, and
(ii) at least about 5% of the therapeutic compound is dissolved from the swallow formulation within 300 seconds at 0 rpm when the dissolution is measured in United States Pharmacopoeia (USP) dissolution apparatus 2 with 900 mL 0.0033 N hydrochloric acid at 37° C.

Typically, when the pH modulating agent of the swallow formulation comprises a base (but no acid), the dissolution rate is greater than 5% at 300 seconds at 0 rpm. More typically the dissolution rate is greater than 20% at 300 seconds at 0 rpm.

Typically when the pH modulating agent of the swallow formulation comprises a base and an acid, the dissolution rate is greater than 5% at 300 seconds at 0 rpm. More typically the dissolution rate is greater than 20% at 300 seconds at 0 rpm.

Preferably, the swallow formulation further comprises an agent which facilitates water uptake. The swallow formulation of the present invention exhibits enhanced dissolution of the therapeutic compound from the formulation.

A dissolution medium comprising 900 mL of 0.0033 N hydrochloric acid contains 3 millimoles of hydrochloric acid, approximating the amount of acid estimated to be present in the residual gastric contents in a fasted subject. This amount of acid can be completely neutralized by high amounts of bases used as pH modulating agents, so that the pH of the dissolution medium will change depending on the levels of pH modulating agents used in a formulation. This is particularly important for investigating the dissolution of drugs where their solubilities is pH dependent.

Dissolution results using 900 mL 0.0033 N hydrochloric acid may be of importance in vivo as the acidity of gastric contents varies significantly, and low acid conditions are associated with the fed and partial prandial states, as well as in patients with suppressed gastric function.

When the stirring speed is reduced to 0 rpm, the dissolution profiles demonstrate the intrinsic characteristics of the fast dissolving formulations of this invention which are able to enhance the dissolution of the drugs without any external stirring. Dissolution results without stirring may be of in vivo significance in conditions where there is gut stasis or reduced gastric activity.

Whilst not wishing to be bound by theory, the bicarbonate assists with the dissolution in a number of ways. Of particular importance is the effervescence, ie the release of CO₂. Whilst it is possible to calculate the theoretical amount of gas that will be produced, it is the rate of production that is critical and it is difficult to measure this directly. The advantage provided by the CO₂ release can be measured indirectly by measuring the rate of dissolution of the tablet without any stirring (ie, 0 rpm). At 0 rpm, the formulation itself will provide the only source of stirring from the gas produced. Use of dissolution media containing lower levels of acid, such as 0.0033 N hydrochloric acid, allows greater discrimination between formulations with different rates of production of carbon dioxide. Formulations which do not effervesce or effervesce only slowly show little if any dissolution even after an extended time.

The preferred amount of pH modulating agent is an amount sufficient to enhance the dissolution of the therapeutic compound from the swallow formulation. This amount will vary depending on the therapeutic compound. Preferably the pH modulating agent will be in an amount so as not to increase the pH of a 900 mL 0.0033 N Hydrochloric acid dissolution medium that contains 3 millimoles of hydrochloric acid to greater than 6.

Examples of suitable active agents include analgesics e.g. opiates and opiate analogs, antipyretics, anti-migraine agents, sedatives, hypnotics, anti-anxiety agents, antipsychotic agents, antidepressants, anticonvulsants, antiemetics, antinauseants, expectorants, antitussives and decongestants, bronchodilators, antihistamines and anti-allergy agents, anti-diarrhoeals, antispasmodics and motility agents, hyperacidity, reflux and ulcer agents, antibiotics, antivirals and antifungals, detoxifying agents and agents used in drug dependence/withdrawal and erectile dysfunction agents.

Preferred therapeutic compounds are those which have one or more base groups such as but not limited to opiates such as hyrocodone, oxycodone, the triptans including eletriptan, rizatriptan, zolmitriptan; the benzodiazepines including diazepam, flurazepam, flunitrazepam, temazepam, alprazolam, lorazepam; fexofenadine; metoclopramide, loperamide, zolpidem, zopiclone, loratadine, ondansetron, granisetron, tadalafil, vardenafil, sildenafil, ranitidine, famotidine, codeine, fentanyl, tramadol, pseudoephedrine, phenylpropanolamine, dextrometthorphan, chlorpheniramine, diphenhydramine, cetirizine, and cimetidine and pharmaceutically acceptable salts thereof.

Preferred therapeutic compounds includes combinations of drugs such as paracetamol and tramadol. Without wishing to be bound by theory, it is believed that certain combinations of drugs may result in synergistic dissolution effects. For example, combination of a base and acid may achieve improved dissolution at lower levels of pH modulsting agent. Again, without wishing to be bound by theory, it is believe that intrinsic micro-stirring in the tablet may effectively promote the dissolution of the lesser soluble drug compared with the mixing achieved as a result of the reaction between the base and the acid (of the pH modulating agent) in the dissolution medium.

In addition, the fast dissolving oral delivery system may contain a combination of pharmaceutically acceptable excipients or other components such as water uptake agents, disintegrants, preservatives, colours, antioxidants, emulsifiers, sweeteners, flavouring agents, binders, glidants and lubricants. In an exemplary form, the fast dissolving delivery system may also contain one or more pharmaceutically active agents. The oral dosage form may be administered by swallowing with water or any other liquid.

Particularly useful active agents include analgesics, anti-allergenics, anti-nausea agents, anti-migraine agents, agents for treating erectile dysfunction and hypnotics.

Another aspect of the invention provides a dosage form such as a coated tablet, uncoated tablet, capsule, powder, paste, cachet, colloid, gel or melt.

### Solubility Considerations

The effect of tablet formulations on drug dissolution will be dependent on the nature and amount of the drug included in each tablet, and the levels of base and acid used in the formulation. The addition of optimised amounts and ratios of acids and bases can significantly improve the dissolution of a range of different drugs as a result of the effect of the couple on the micro pH in the tablet and on the pH of the dissolution medium, which in turn increases the solubility of a drug.

In general,
for basic drugs where the solubility decreases with an increase in pH, then for maximum dissolution, it is important that there is no significant net increase in the pH such as is achieved with stoichiometric amounts of acid and a base (in the pH modulating agent) since they react with each other. There will only be a net effect on pH if one or the other component is in excess.
amphoteric drugs behave like basic or acidic drugs depending on the pKa and the pH.

Amphoteric drugs behaving as bases will demonstrate reduced solubility at higher pH as the more soluble acid salt is converted to the less soluble, less ionised form. As these drugs also behave as acids it is important to optimise the pH in the formulation for optimum solubility for each specific drug.

The present invention further contemplates a method for delivering a therapeutic compound that is a base, a salt of a base, an amphoteric compound or a salt of an amphoteric compound by oral delivery including administration such as by swallowing, the method comprising orally delivery, including administering, a formulation comprising a therapeutic compound with an appropriate amount of one or more pH modulating agents wherein at least one of the pH modulating agents is a carbonate so as to enhance the dissolution of the therapeutic compound from the swallow formulation.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be understood that unless otherwise indicated, the subject intervention is not limited to specific formulation components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise. Thus, for example, reference to “therapeutic compound” includes a single therapeutic compound, as well as two or more therapeutic compounds; reference to “a pH modulating agent” includes a single pH modulating agent, as well as two or more pH modulating agents; reference to a “water uptake agent” includes a single water uptake agent, as well as two or more water uptake agents; and so forth.

In describing and claiming the present invention, the following terminology is used in accordance with the definitions set forth below.

Where used herein “unionized” refers to a drug that is largely unionized between approximately pH 2 and pH 8. Of course the person skilled in the art will understand that there will be some extent of unionization of almost any drug in the right pH. Typically the unionized drug will be >50% ionised below pH 2 and above pH 8.

A “swallow formulation” is any formulation which is administered to a subject by the action of swallowing the dosage form intact. The dosage form comprising the swallow formulation may be a coated tablet or capsule which does not have the same dissolution characteristics of the swallow formulation contained therein.

The terms “therapeutic compound”, “compound”, “pharmacologically active agent”, “medicament”, “active”, “active ingredient”, “drug” and “drug component” are used interchangeably throughout this specification. The terms also encompass pharmaceutically acceptable and pharmacologically active ingredients of those active agents specifically mentioned herein including but not limited to salts, esters, amides, pro-drugs, active metabolites, analogs and the like. When the terms “active agent”, “compound”, “pharmacologically active agent”, “medicament”, “active”, “drug”, and “drug component” are used, then it is to be understood that this includes those compounds per se as well as pharmaceutically acceptable, pharmacologically active salts, esters, amides, pro-drugs, metabolites, analogs, etc. The terms “agent”, “compound” etc may be a single molecule or a composite of molecules.

By the term “effective amount” or “therapeutically effective amount” of a therapeutic compound as used herein means that a sufficient amount of a therapeutic compound is used to provide the desired therapeutic effect or the desired physiological or biochemical event including the amelioration of symptoms being treated or prevented. Of course, undesirable effects, e.g. side effects, are sometimes manifested along with the desired therapeutic effect; hence, a practitioner balances the potential benefits against the potential risks in determining what is an appropriate “effective amount”.

The terms “delivery” and “administration” are used interchangeably throughout the specification to mean the act of providing the oral dosage form to an individual. The term “administering” is considered herein synonymous with “delivering”, “providing”, “introducing” or “swallowing”.

By “pharmaceutically acceptable excipient” is meant a pharmaceutical vehicle comprised of a material that is not biologically or otherwise undesirable, i.e. the oral dosage form may be administered to a subject along with a therapeutic compound without causing any or a substantial adverse reaction. Excipients may include carriers and other additives such as diluents, binders, detergents, colouring agents, flavouring agents, wetting or emulsifying agents, preservatives, glidants, lubricants and the like as well as disintegrants.

The terms “treating” and “treatment” as used herein refer to reduction or amelioration in severity and/or frequency of symptoms, elimination of symptoms and/or underlying cause and/or prevention of the occurrence of symptoms and/or their underlying cause. Thus, for example, “treating” a patient involves prevention of a particular disorder or adverse physiological event in a susceptible individual as well as treatment of a clinically symptomatic individual by inhibiting or causing regression of a particular condition. Thus, for example, a method of treating a patient in need of pain relief encompasses both prevention of pain as well as treating conditions of pain.

“Patient” as used herein refers to an animal, preferably a mammal and more preferably human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host or recipient. The compounds and methods of the present invention have applications in human medicine, veterinary medicine as well as in general, domestic or wild animal husbandry. For convenience, an “animal” includes an avian species such as a poultry bird, an aviary bird or game bird.

The preferred animals are humans or other primates, livestock animals, laboratory test animals, companion animals or captive wild animals. A human is the most preferred target.

A “pH modulating agent” includes one or more than one pH modulating agents which alter the pH of an aqueous solution. These may include acids, bases or a combination of one or more and/or bases.

The carbonate may be any pharmaceutically acceptable carbonate or a mixture thereof. Reference to a “carbonate” includes a single agent or multiple (ie. two or more) agents. Preferred carbonates include but are not limited to sodium carbonate, sodium bicarbonate, calcium carbonate, magnesium carbonate, ammonium carbonate, ammonium bicarbonate, potassium bicarbonate, sodium glycine carbonate, disodium glycine carbonate, arginine carbonate, lysine carbonate and/or other pharmaceutically acceptable carbonates or homologs or functional equivalents thereof and combinations thereof.

Other pH modulating agents may be pharmaceutically acceptable acids or acidic salts including citric acid, tartaric acid, succinic acid, ascorbic acid, malic acid, fumaric acid, metatartaric acid, adipic acid, sodium acid citrate, potassium acid citrate, glycine citrate, potassium acid tartrate, sodium acid tartrate, aspartic acid, glutamic acid, glycine, leucine, tyrosine, tryptophan, glycine fumarate, glycine hydrochloride, monophosphate glycine and combinations thereof.

A “water uptake agent” is any agent which will facilitate the uptake of water by absorbing, dissolving in or wicking water, used alone or in combination. These may include wicking agents, disintegrants, binders, carriers and other hydrophilic excipients. Generally, but not exclusively, a “water uptake agent” facilitates uptake of water into the swallow formulation.

Preferably the carbonate is present in an amount from about 1% to about 45% by weight of swallow formulation and in an amount that will neutralise between 0.01 and 9.0 millimoles of hydrochloric acid. More preferably the carbonate is present in an amount from about 1% to about 40% by weight in the swallow formulation and in an amount that will neutralise between 0.02 and 8.0 millimoles of hydrochloric acid.

Examples of particular amounts of carbonate include 1 to 50% by weight of the swallow formulation.

Conveniently the carbonate component in the pH modulating agent is present in an amount from about 1 mg to about 450 mg in the swallow formulation.

Examples of particular amounts of carbonate include 1 mg to 450 mg per swallow formulation. More preferably the carbonate is present in an amount from about 2 mg to 400 mg. Preferably at least one of the carbonates is soluble and/or dispersible.

Examples of suitable carbonates include, without being limited to sodium carbonate, sodium bicarbonate, calcium carbonate, magnesium carbonate, ammonium carbonate, ammonium bicarbonate, potassium bicarbonate, sodium glycine carbonate, disodium glycine carbonate, arginine carbonate, lysine carbonate and/or other pharmaceutically acceptable carbonates or homologs or functional equivalents thereof and combinations thereof.

Preferably, the carbonates of the swallow formulation are soluble and/or dispersible carbonates such as sodium bicarbonate or potassium bicarbonate or magnesium carbonate or combinations thereof.

Optionally the swallow formulation may contain further pH modulating agents such as pharmaceutically acceptable acids or acidic salts including citric acid, tartaric acid, succinic acid, ascorbic acid, malic acid, fumaric acid, metatartaric acid, adipic acid, sodium acid citrate, potassium acid citrate, glycine citrate, potassium acid tartrate, sodium acid tartrate, aspartic acid, glutamic acid, glycine, leucine, tyrosine, tryptophan, glycine fumarate, glycine hydrochloride, monophosphate glycine and combinations thereof.

In one swallow formulation embodiment the carbonate is sodium bicarbonate and/or potassium bicarbonate and/or magnesium carbonate and is present in an amount from about 1% to 50% by weight of the swallow formulation.

Suitable water uptake agents include cross-linked polyvinylpyrrolidone (crospovidone), croscarmellose sodium, sodium starch glycolate, starch, starch derivatives, hydroxypropylcellulose, low substituted hydroxypropylcellulose, hydroxypropylmethylcellulose, alginic acid, sodium alginate, calcium sulphate, calcium carboxymethylcellulose, microcrystalline cellulose, powdered cellulose, colloidal silicon dioxide, docusate sodium, guar gum, magnesium aluminium silicate, methylcellulose, polacrilin potassium, silicified microcrystalline cellulose, magnesium oxide, tragacanth, mannitol, sorbitol, xylitol, sucrose, lactose, fructose, maltose, polyethylene glycol, aminoacids, cyclodextrin, urea and/or polyvinylpyrrolidone (povidone, PVP).

The water uptake agent may be present in an amount from 5% to 95% by weight of the swallow formulation and more preferably between 10% and 90% by weight of the swallow formulation.

Preferably, the ratio of water uptake agent to pH modulating agent is between 0.1:1 and 20:1 by weight such as 0.1:1, 0.2:1, 0.3:1, 0.4:1, 0.5:1, 0.6:1, 0.7:1, 0.8:1, 0.8:1, 0.9:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1. More preferably the ratio of water uptake agent to pH modulating agent is between 0.3:1 and 15:1 by weight.

In one embodiment at least 80% of the therapeutic compound is dissolved from the swallow formulation within 180 seconds in USP dissolution apparatus 2 with 900 mL 0.0033 N hydrochloric acid at 30 rpm and 37° C. Even more preferably, at lest 90% is dissolved in 180 seconds.

In another embodiment at least 70% of the therapeutic compound is dissolved from the swallow formulation within 120 seconds in USP dissolution apparatus 2 with 900 mL 0.0033 N hydrochloric acid at 30 rpm and 37° C. Even more preferably, at least 80% is dissolved in 120 seconds and even more preferably, at least 90% is dissolved in 120 seconds.

In one embodiment of the invention the therapeutic compound is a basic compound chosen from the group comprising zolmitriptan, alprazolam, lorazepam, diazepam or combinations thereof. Preferably the carbonate is present in an amount between 1% and 50% by weight of the swallow formulation. More preferably the carbonate is present in an amount between 1% and 40%. Preferably the carbonate is present in an amount between 1 mg and 450 mg or more preferably in an amount between 1 mg and 350 mg. Preferably the carbonate is sodium bicarbonate. Optionally the swallow formulation further comprises up to 50% by weight of a pharmaceutically acceptable acid.

In another embodiment of the invention the therapeutic compound is a salt of a basic therapeutic compound chosen from the group comprising sildenafil citrate, pseudoephedrine hydrochloride, eletriptan hydrobromide, rizatripan benzoate, metoclopramide hydrochloride, loperamide hydrochloride, codeine phosphate, tramadol hydrochloride, zolpidem tartrate, ondansetron hydrochloride or combinations thereof. Preferably the carbonate is present in an amount between 1% and 50% by weight of the swallow formulation. More preferably the carbonate is present in an amount between 1% and 40% by weight of the swallow formulation. Preferably the carbonate is present in an amount between 1 mg and 450 mg, more preferably and amount between 1 mg and 350 mg. Preferably the carbonate is a bicarbonate such as sodium bicarbonate or potassium bicarbonate or a mixture thereof. Optionally the swallow formulation further comprises up to 50% by weight of a pharmaceutically acceptable acid.

In another embodiment the therapeutic compound is an amphoteric compound chosen from the group consisting of cetirizine, lorazepam or combinations thereof. Preferably the carbonate is present in an amount between 1% and 50% by weight of the swallow formulation. More preferably the carbonate is present in an amount between 1% and 40% by weight of the swallow formulation. Preferably the carbonate is present in an amount between 1 mg and 450 mg or more preferably in an amount between 1 mg and 300 mg. Preferably the carbonate is sodium bicarbonate. Optionally the swallow formulation may comprise up to 50% by weight of a pharmaceutically acceptable acid such as tartaric acid.

In another embodiment the therapeutic compound is a salt of an amphoteric compound chosen from the group consisting of fexofenadine hydrochloride, cetirizine hydrochloride or combinations thereof. Preferably the carbonate is present in an amount between 1% and 50% by weight of the swallow formulation. Preferably the carbonate is present in an amount between 1% and 40% by weight of the swallow formulation or more preferably in an amount between 1 mg and 300 mg. More preferably the carbonate is present in an amount between 1 mg and 450 mg. Preferably the carbonate is sodium bicarbonate. Optionally the swallow formulation may comprise up to 50% by weight of a pharmaceutically acceptable acid such as tartaric acid.

Optionally the swallow formulation may also comprise one or more pharmaceutically acceptable excipients or other components such as carries, glidants, emulsifiers, diluents, binders, preservatives, wicking agents and/or disintegrants.

The swallow formulation may further contain flavouring agents, colouring agents and sweeteners.

In one embodiment the swallow formulation is co-administered with an aqueous fluid such as water. The co-administered fluid may be administered, before, after or with the swallow formulation.

Another aspect of the present invention is directed to a swallow formulation comprising a therapeutic compound that is a base, a salt of a base or an amphoteric compound or a salt of an amphoteric compound with an appropriate amount of one or more pH modulating agents wherein at least one pH modulating agent is a carbonate and which permits at least about 70% of the therapeutic compound to dissolve from the swallow formulation within 180 seconds in USP dissolution apparatus 2 with 900 mL N hydrochloric acid at 30 rpm and 37° C. said dosage form further comprising one or more pharmaceutically acceptable carriers, diluents and/or excipients, wherein the swallow formulation is co-administered with fluid.

The swallow formulation may comprise one, two, three or more therapeutic agents. Accordingly, in one preferred embodiment, the present invention provides a swallow formulation comprising two or more therapeutic compounds and one or more carbonates in an appropriate amount wherein at least one of the therapeutic compounds is a base, a salt of a base or an amphoteric compound or a salt of an amphoteric compound and at least 70% dissolves from the swallow formulation within 180 seconds in USP dissolution apparatus 2 with 900 mL 0.0033 N hydrochloric acid at 30 rpm and 37° C.

In another aspect of the present invention there is provided a dosage form comprising a swallow formulation comprising a therapeutic compound that is a base, a salt of a base or an amphoteric compound or a sale of an amphoteric compound and one or more pH modulating agents wherein at least one pH modulating agent is a carbonate in an amount from 1% to about 50% by weight of the swallow formulation and wherein at least about 70% of the therapeutic compound is dissolved from the swallow formulation within 180 seconds in USP dissolution apparatus 2 with 900 mL 0.0033 N hydrochloric acid at 30 rpm and 37° C.

The dosage form may be a tablet, capsule, powder, cachet, paste, colloid, gel or melt. The does form may optionally be in a chewable form.

The dosage form of the invention may be coated, uncoated and/or layered tablet. Suitable coatings include water soluble polymer based coatings such as, povidone or hypromelloside. Suitable coating polymers may also be a derivative of cellulose (cellulose acetophthalate, hypromellose phthalate) or a derivative of an acrylic polymer (methacrylate acid copolymer). Optionally, the dosage form may be coated with gelatine.

The dosage form may contain one or more further pharmaceutically active agents. In one embodiment, the dosage form is a multi-phase release dosage form containing a further therapeutic compound having a dissolution of less than 70% in 180 seconds in USP dissolution apparatus 2 with 900 mL 0.0033 N hydrochloric acid at 30 rpm and 37° C.

Swallow formulations of the present invention may be manufactured by admixing the ingredients simultaneously or sequentially and then converting into a dosage unit such as a tablet, capsule, wafer or the like.

Tablets of the present invention may be manufactured by and convenient manufacturing method known to the person skilled in the relevant technology including for example, manufacture by direct compression or granulation and compression.

The present invention further contemplates a method for the amelioration or prevention of the symptoms associated with a disease or disorder, including pain, fever, discomfort, migraine, nausea, insomnia, sleep disorders, allergic rhinitis, atopy and erectile dysfunction in a subject, the method comprising administering to said subject a swallow formulation comprising a therapeutic compound that is a base, a sale of a base or an amphoteric compound or a sale of an amphoteric compound and one or more pH modulating agents wherein at least one of the pH modulating agents is a carbonate in an amount between 1% and 50% by weight of the swallow formulation, the therapeutic compound having enhanced dissolution from the swallow formulation, the administration being for a time and under conditions to prevent or ameliorate symptoms of the condition.

Conditions contemplated herein include any condition associated with a disease or disorder in need of treatment. Conditions include but are not limited to conditions associated with pain and/or fever, with the central nervous system, alimentary system, cardiovascular system, musculoskeletal system, respiratory system, allergy and immune system and genitourinary system, microbial infections, conditions requiring hormonal and steroidal treatment and conditions associated with the metabolism.

Another aspect of the present invention contemplates a method for management if a condition in a subject experiencing the condition or anticipating to experience the condition, said method comprising administering to said subject an oral delivery system comprising a therapeutic compound that is a base, a sale of a base, an amphoteric compound or a salt of an amphoteric compound to treat the condition and one or more pH modulating agents wherein at least of the pH modulating agents is a carbonate in an amount between 1% and 50% by weight of the swallow formulation, the therapeutic compound having enhanced dissolution from the swallow formulation, the administration being for a time and under conditions to prevent or ameliorate symptoms of the condition.

These methods also involve the oral dosage from having one or more pharmaceutically acceptable excipients.

As indicated above, the present invention extends to human, veterinary and animal husbandry applications.

### EXAMPLES

The present invention is further described by the following non-limiting examples which relate to the following basic and amphoteric actives:
1 Fexofenadine hydrochloride 180 mg (salt of amphoteric drug)
2 Pseudoephedrine hydrochloride 60 mg (salt of basic drug)
3 Eletriptan hydrobromide 40 mg (base) (salt of basic drug)
4 Rizatriptan benzoate 14.53 mg (salt of basic drug)
5 Metoclopramide hydrochloride 10 mg (salt of basic drug)
6 Loperamide hydrochloride 2 mg (salt of basic drug)
7 Codeine phosphate 30 mg (salt of basic drug)
8 Tramadol hydrochloride 37.5 mg (salt of basic drug)
9 Diazepam 5 mg (basic)
10 Lorazepam 2.5 mg (amphoteric)
11 Alprazolam 1 mg (basic)
12 Sildenafil citrate 140 mg (salt of a basic drug)
13 Ondansetron hydrochloride 10 mg (salt of a basic drug)
14 Zolmitriptan 2.5 mg (base)
15 Zolpidem tartrate 10 mg (salt of a basic drug)
16 Cetirizine hydrochloride 10 mg (salt of an amphoteric drug)
17 Comparative Data for Examples 1 to 16
18 Tramadol Hydrochloride 37.5 mg (salt of a base) with Paracetamol 325 mg (acid)
   Examples 1 to 11 include dissolution profiles for:
a formulation designated 1 containing bicarbonate alone in accordance with the present invention measured at 30 rpm and 0 rpm,
a formulation designated 2 containing bicarbonate & acid in accordance with the present invention measured at 30 rpm and 0 rpm, and
a commercial product without bicarbonate sourced in Australia or USA as a comparative example measured at 30 rpm only.

Example 18 includes dissolution profiles for paracetamol and tramadol hydrochloride formulations including:
one formulation according to the invention with bicarbonate alone designated 1,
a second formulation according to the invention containing sodium bicarbonate with fumaric acid as an example of an organic acid designated 2, and
a commercial product which contains no pH modulating agents as well as any fast dissolving commercial products containing pH modulating agents.

For Example 18, dissolution data are provided using USP dissolution apparatus 2 at 37° C. and 0.0033 N hydrochloric acid at 30 rpm and 0 rpm. This dissolution medium discriminates better between formulations designed for fast dissolution than more acidic dissolution media where the effect of high acid concentration tends to mask formulation effects on dissolution.

For the dissolution profiles, drug concentrations were measured by UV absorbance at an appropriate wavelength for each drug using flow through cells for automatic continuous sampling.

### Overview of Examples and Methods of Preparation

Examples 1 and 16 are salts of amphoteric drugs. Examples 2 to 8, 12, 13 and 15 are salts of basic drugs. Examples 9, 10 and 14 are bases. Example 11 is an amphoteric drug. Example 18 is a combination of a salt of a basic drug with an acidic drug.

In Examples 4 and 8 and 13 to 16, the formulations were prepared by dry blending the ingredients prior to compression unless otherwise stated. The powder blend was compressed with suitable size tooling on a rotary press to produce tablets with hardness in the range 3-12 Kp.

Examples 12 and 18 were prepared as indicated below under their respective headings.

Formulations of drugs for Examples 1 to 3, 5 to 7 and 9 to 11 were prepared by crushing commercially available product in a mortar and pestle, and where applicable passing through a 280 μm screen to remove any residual film coating. The resultant powder was dry blended proportionally with other ingredients before compression.

In Examples 12 to 16 the formulations designated ‘1’ are always the comparative examples containing no carbonate. All other formulations of Examples 12 to 16 contain carbonate in accordance with the present invention.

While the complete quantitative formulations of the commercial products are not known, all ingredients contained in the products are listed in the product information available from the manufacturer. The amount of drug and any actives are quantified, but only the presence or absence of other ingredients is known. In the tables for these examples, the symbol “√” indicates that a specific ingredient is present, and will be present at the same level in all formulations provided.

Although these formulations have not been optimised, they do demonstrate the applicability of the present invention to a range of different basic compounds, salts of basic compounds and amphoteric compounds.

### Dissolution Testing

The following USP dissolution apparatus II with 1000 mL dissolution vessels and paddle stirrers was used to perform the dissolution testing:
VanKel VK 7010 Dissolution bath
VanKel VK 750 D Heater/Circulator
Gilson Minipulse peristaltic pump for automatic continuous sampling
   All testing was conducted in a dissolution medium containing 900 mL of 0.0033 N hydrochloric acid at 37° C. which is effective in discriminating between fast dissolving formulations. 900 mL of this medium contains the absolute amount of acid estimated to be present in the gastric contents in vivo, namely 3 millimoles, and its pH will change when high levels of sodium bicarbonate used in some formulations are added.

The materials used for preparation of dissolution media were:
32% w/w concentrated hydrochloric acid (HCl) (AR quality from Rowe Scientific)
RO water from in-house Millipore Elix® water system.

Dissolution results were measured as the mean of 2 replicates. Repeating the testing at 0 rpm provides more discrimination between formulations.

For measurements at 30 rpm, after 20 minutes, the stirring speed was increased to demonstrate the extent of further dissolution that could be achieved.

Drug concentrations for Examples 1 to 16 were measured using a Varian Cary 50 UV-Vis Spectrophotometer set at an appropriate wavelength. For each drug, the optimal wavelength was selected after running UV scans in the dissolution medium. For drug concentration measurements at 0 rpm, the amount of dissolution medium bled off was so small as to have negligible effect on the main body of dissolution media.

Drug concentrations of the paracetamol/tramadol formulations of Example 18 were measured by HPLC analysis for each drug in samples taken at selected intervals.

Solution pH was measured using a TPS WP81 pH, Salinity, Temperature & Conductivity Meter.

### Overview of Results

Table 1 summarises the dissolution results for Examples 1 to 11 at 30 rpm.

Tables 2 and 2a summarise the dissolution results at 0 rpm for the drugs exemplified in Examples 1 to 16 of the specification, where the pH modulating agent comprises a base and an acid, and a base alone, respectively.

Tables 3 to 46 and 49 to 52 set out the formulations of the examples and their corresponding dissolution rates.

Table 47 presents the consolidated dissolution data on all drugs exemplified in Examples 1 to 16 of the specification.

Table 48 provides a summary of aqueous solubility data for all drugs exemplified in the patent application.

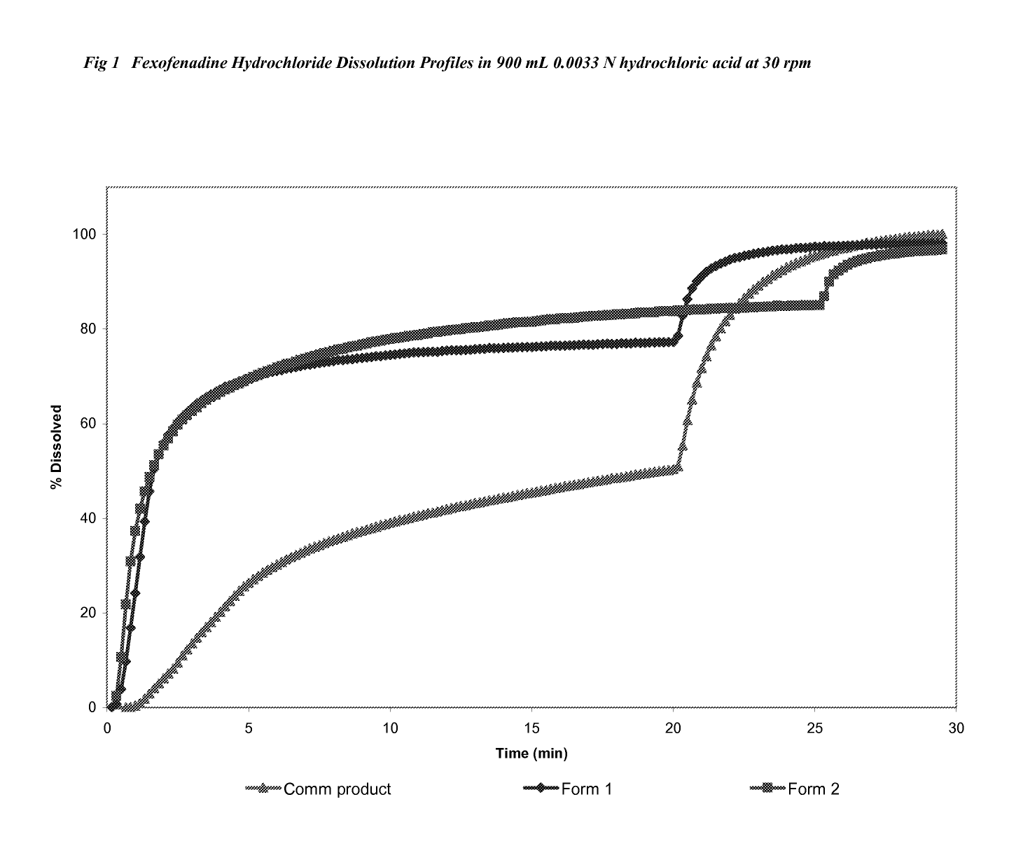

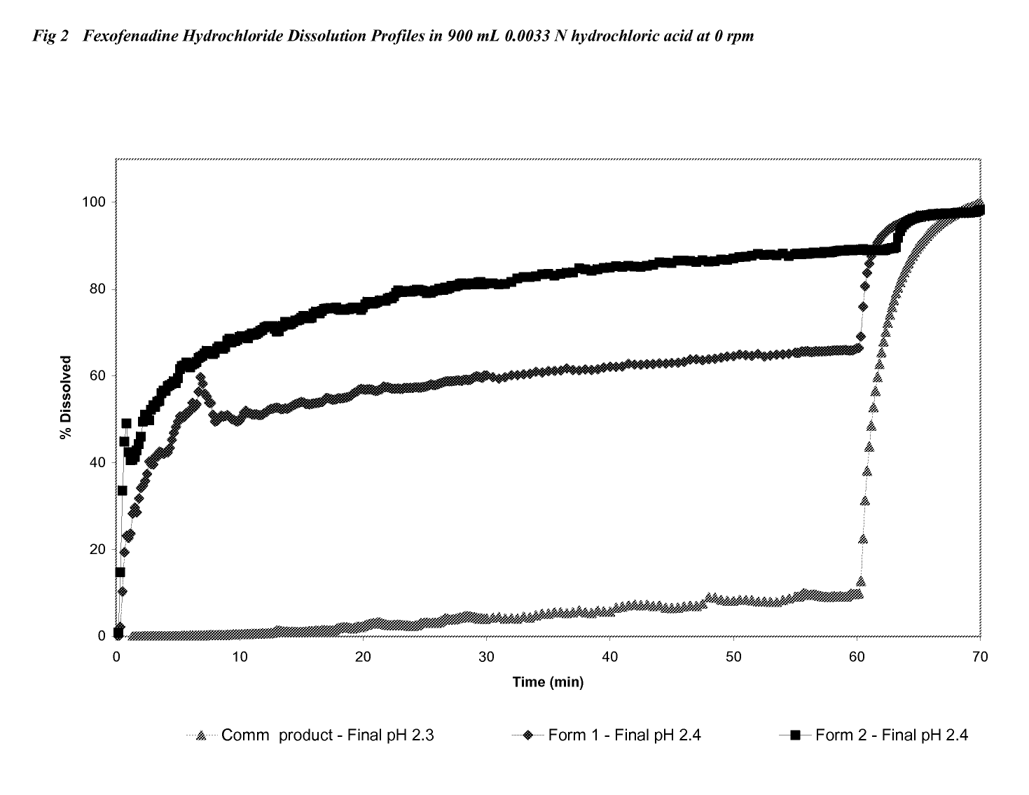

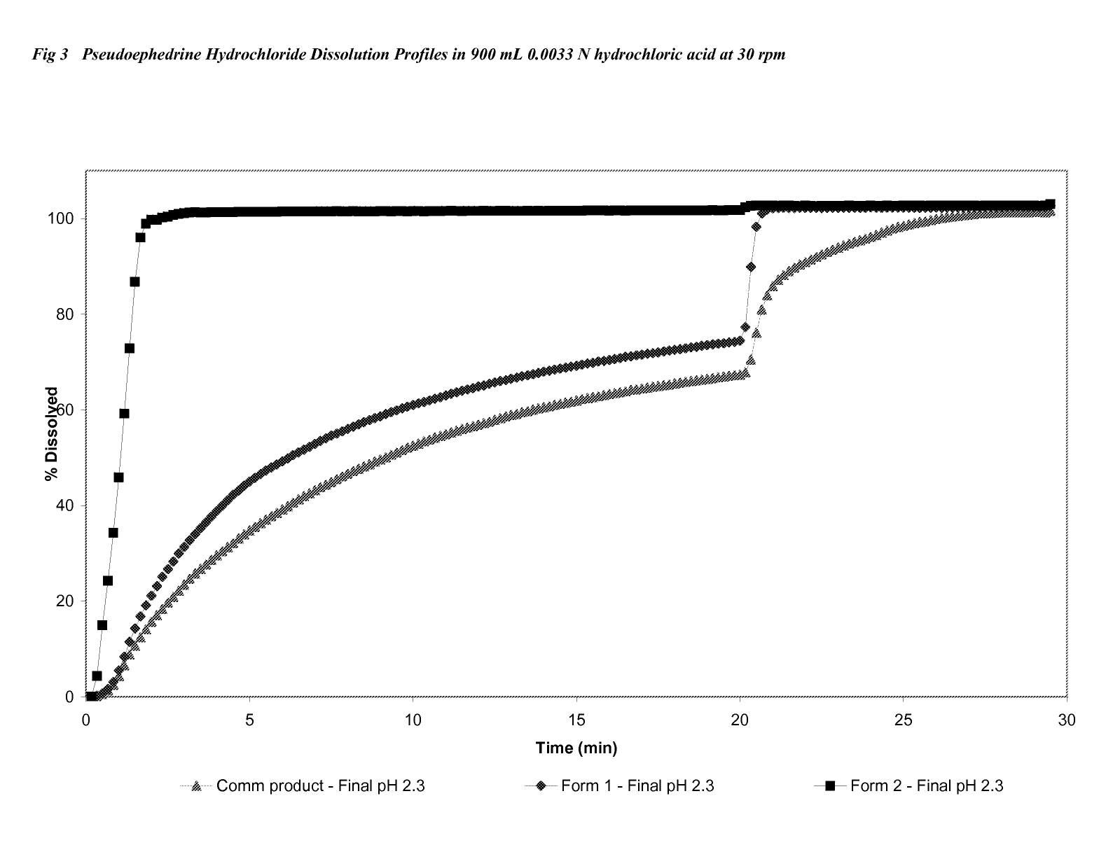

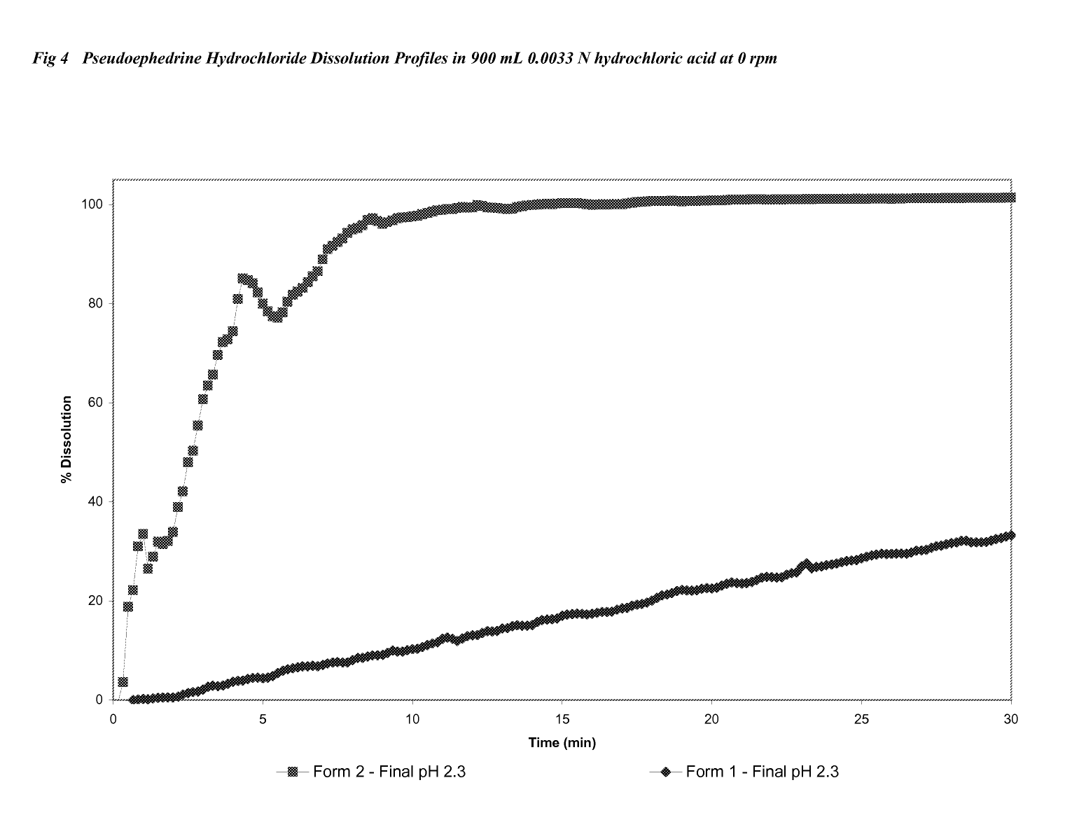

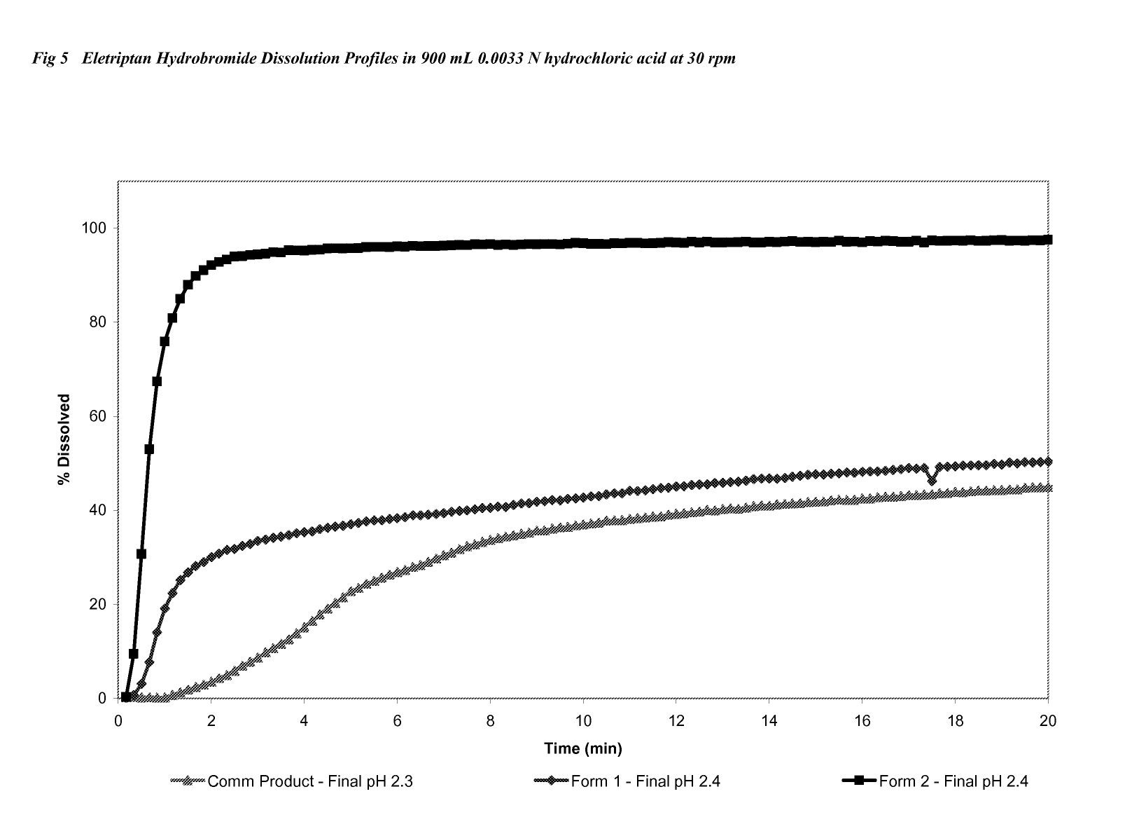

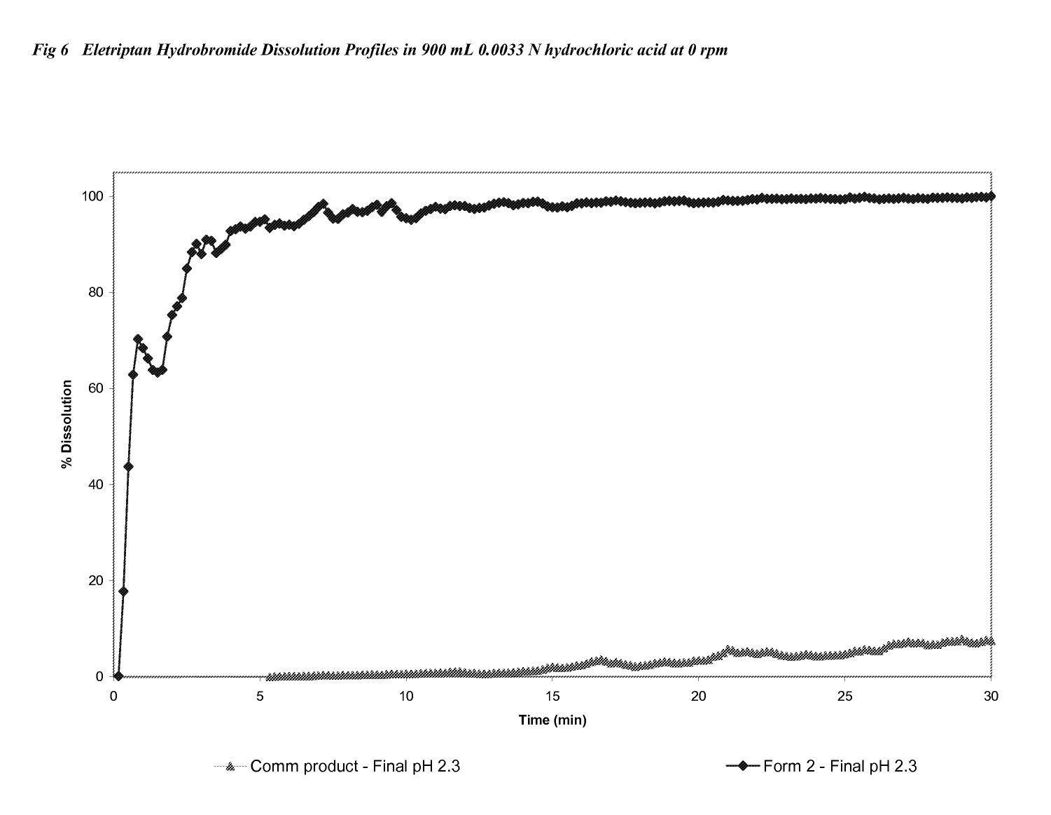

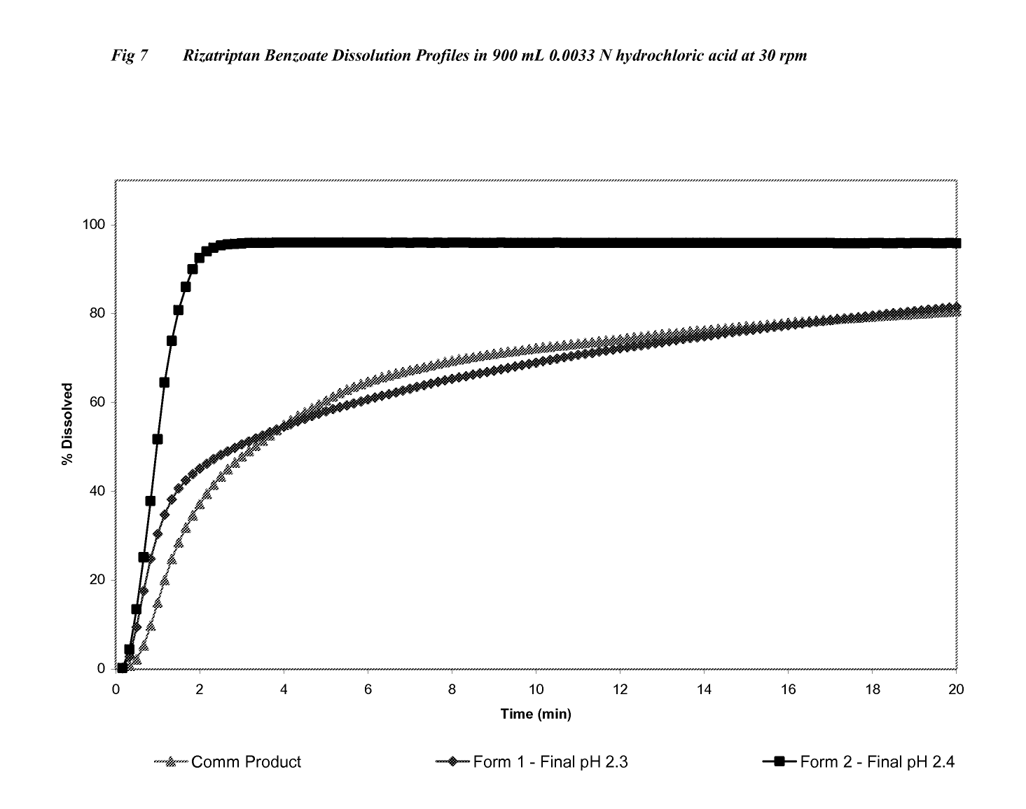

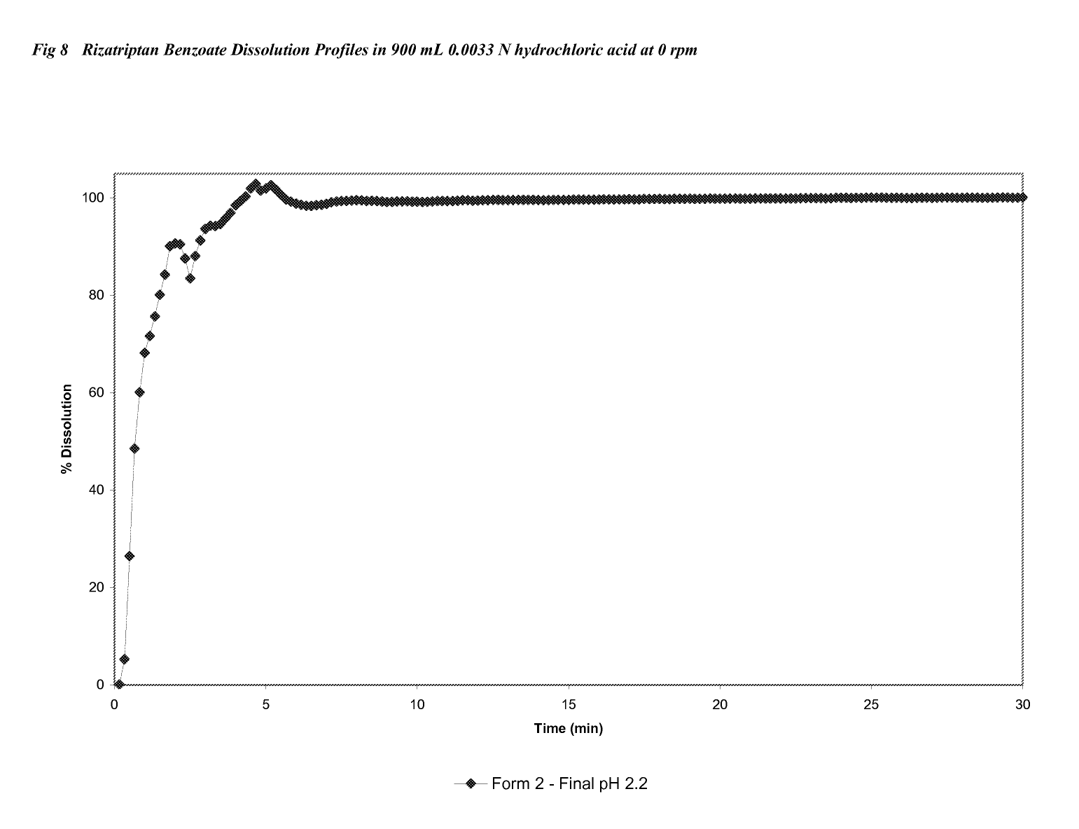

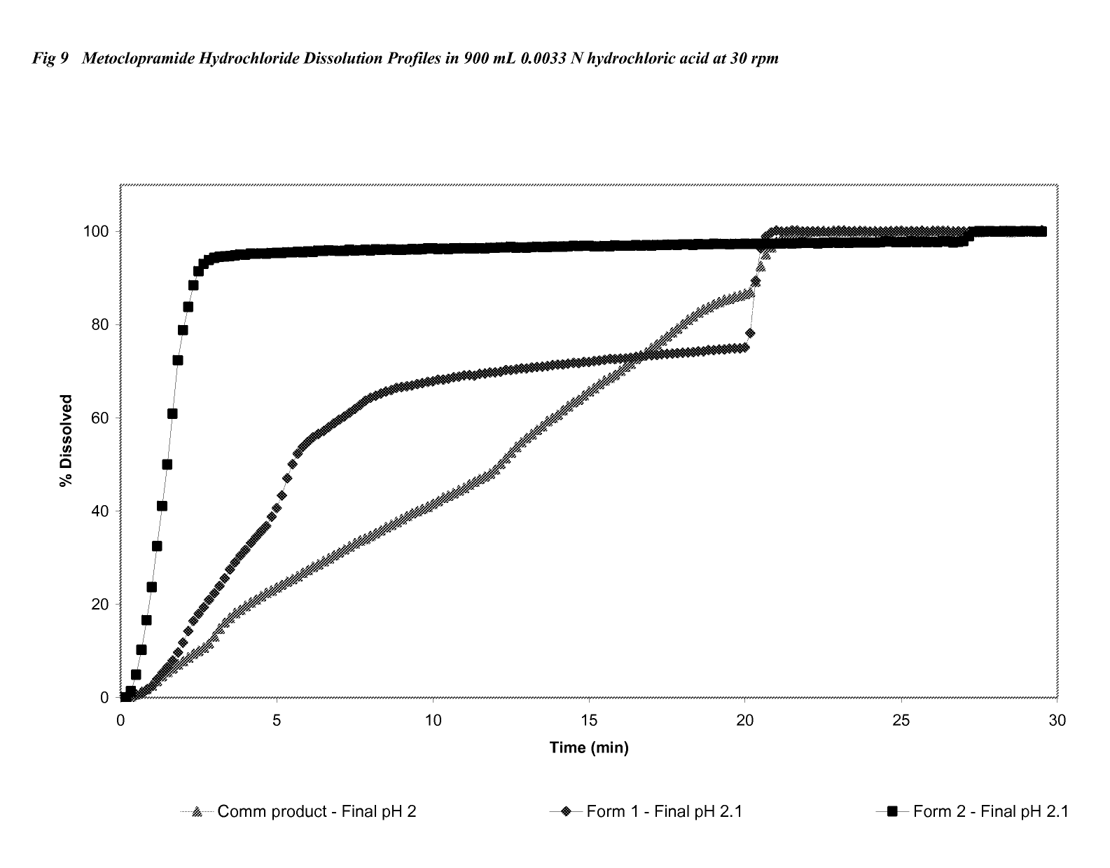

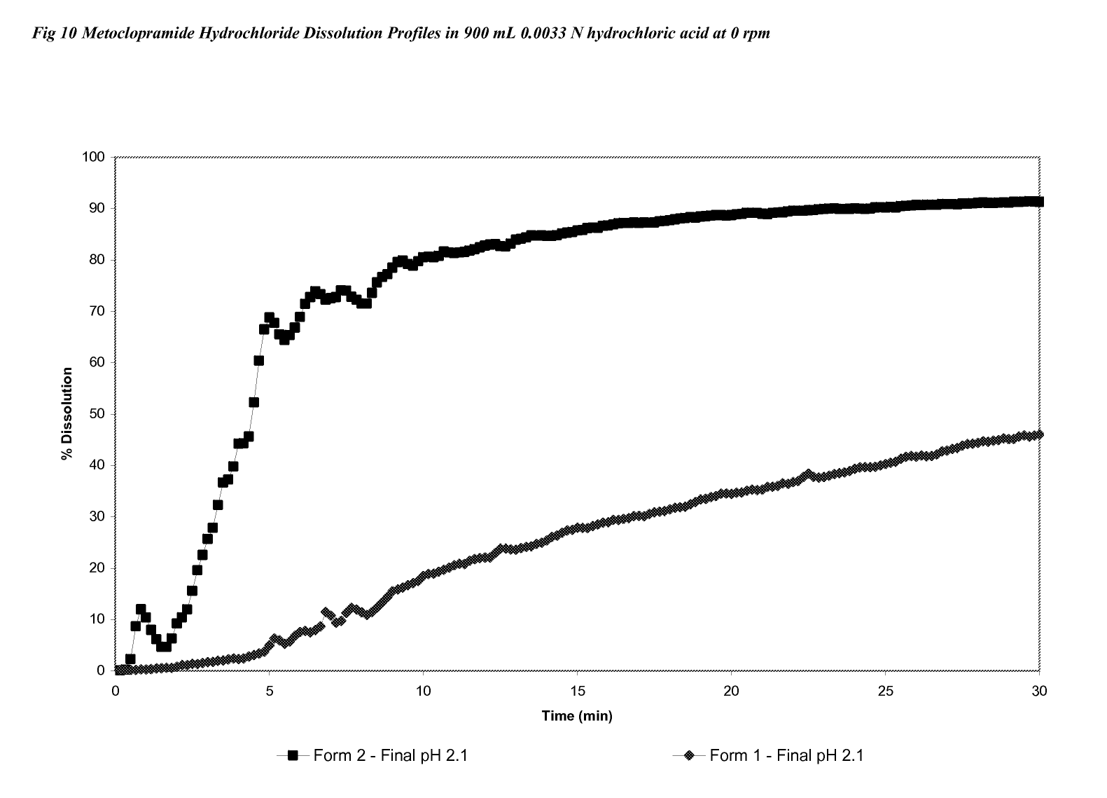

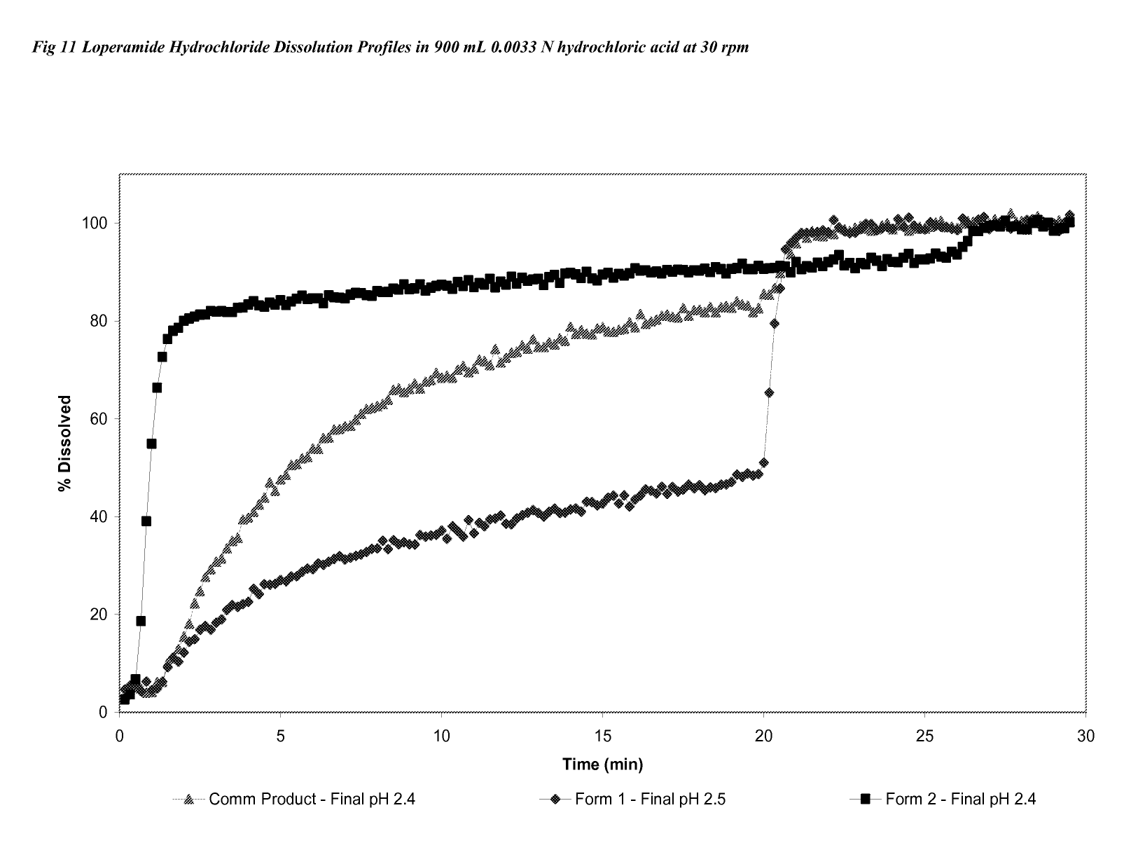

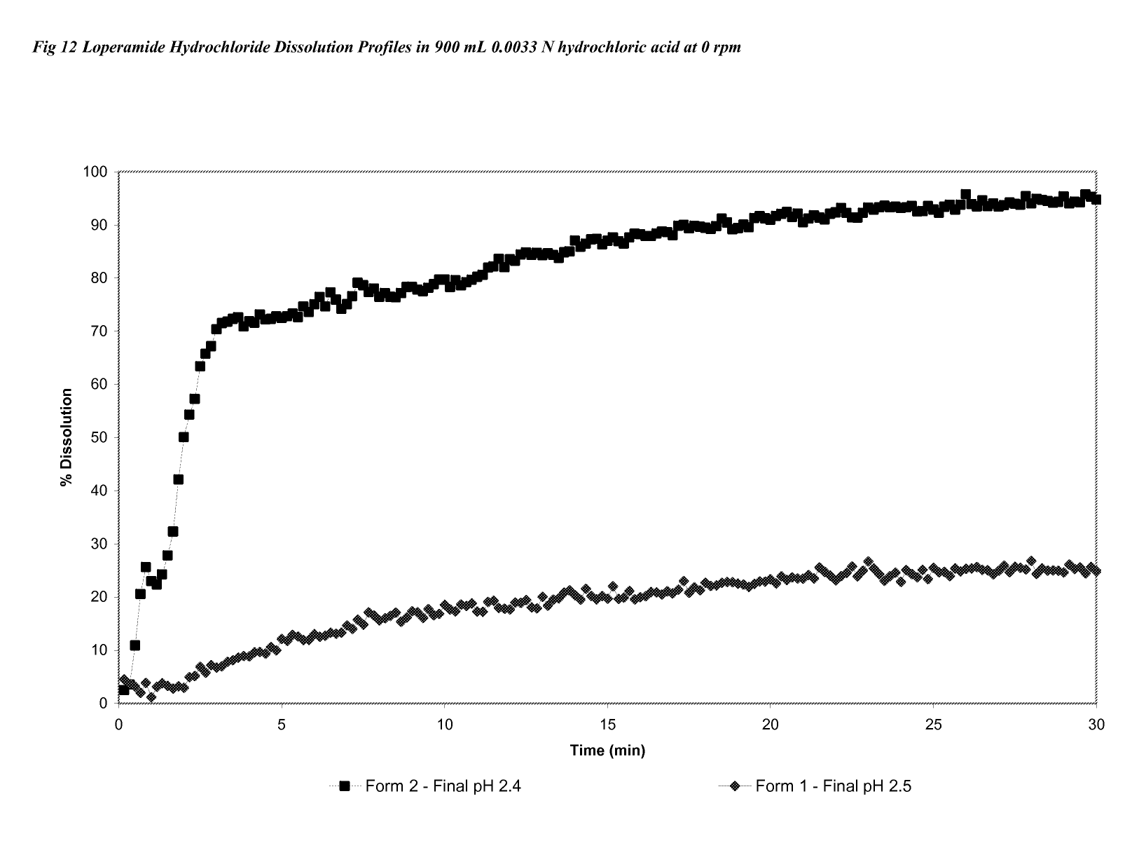

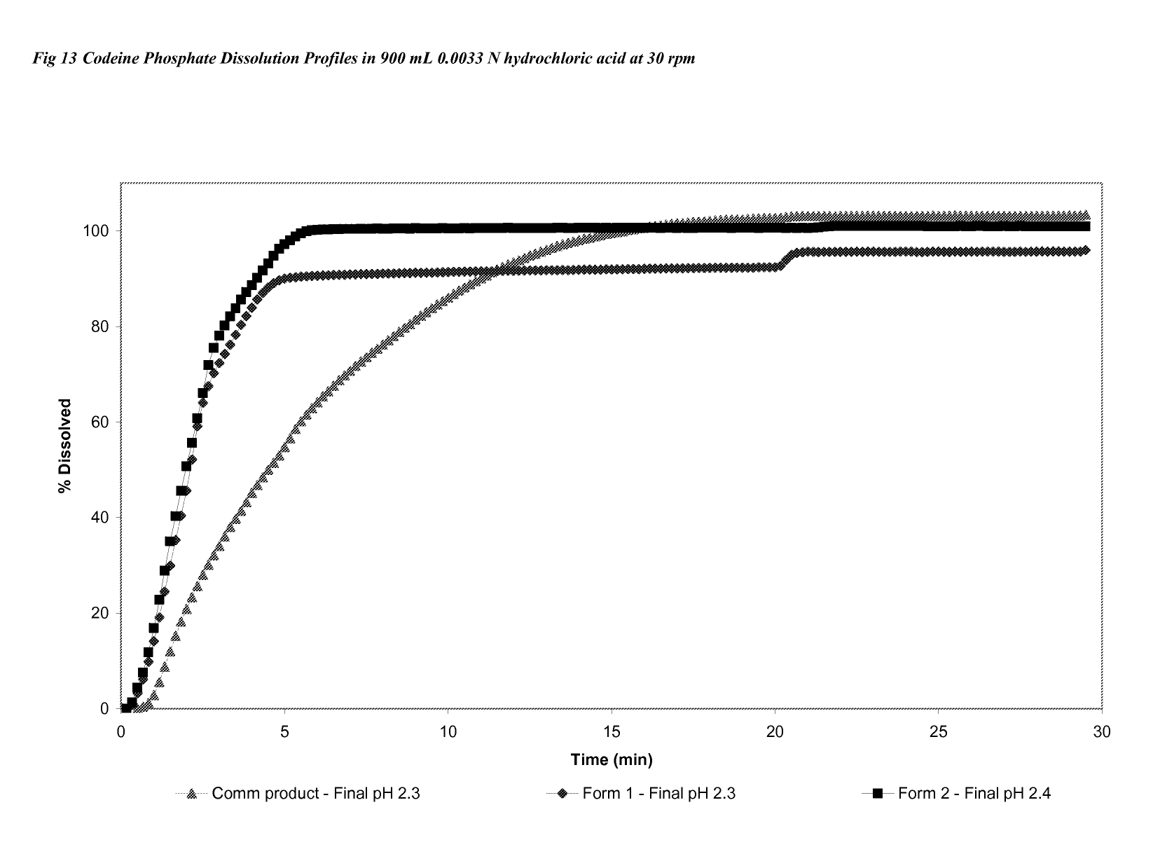

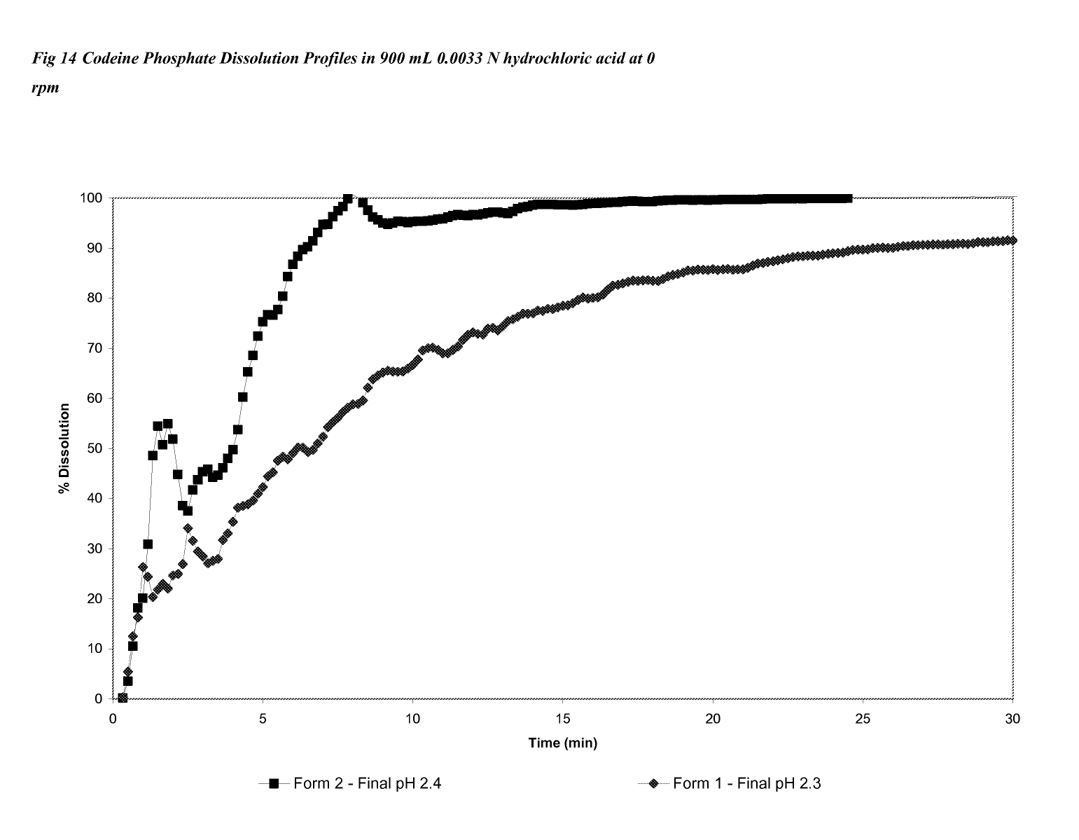

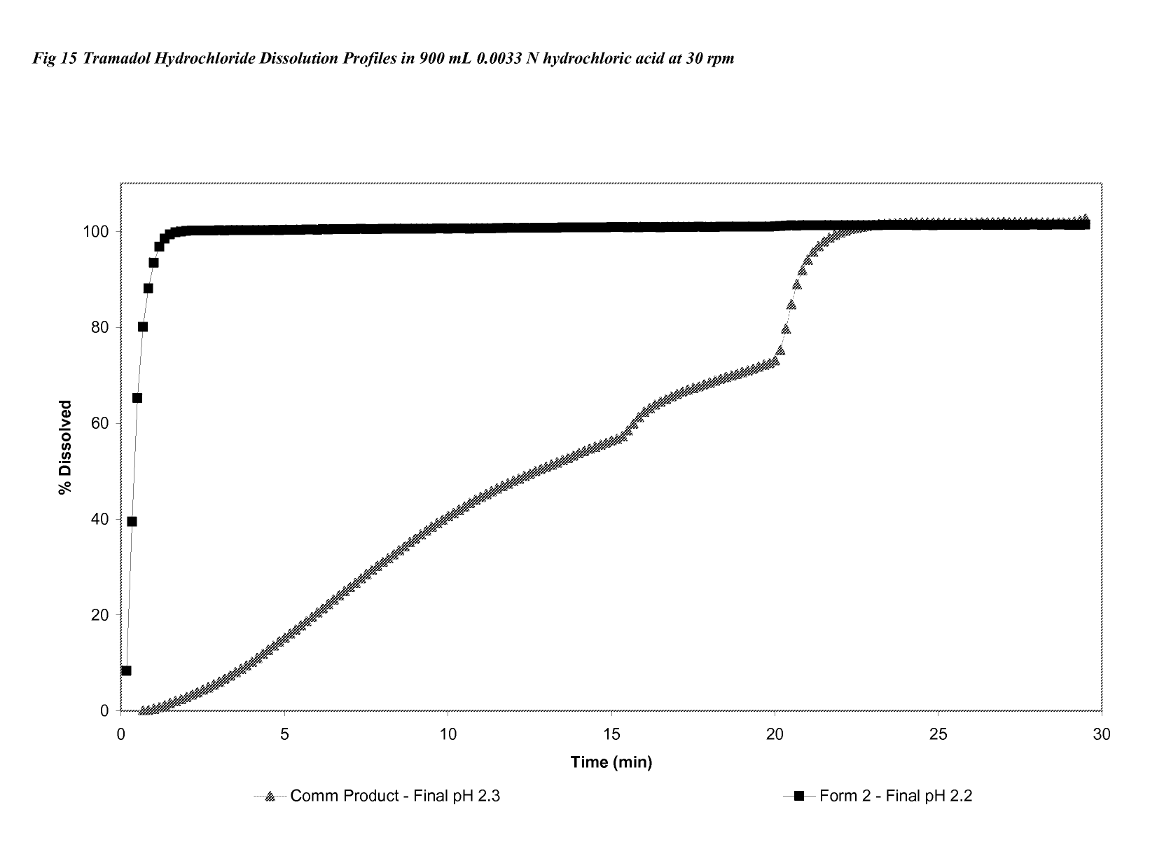

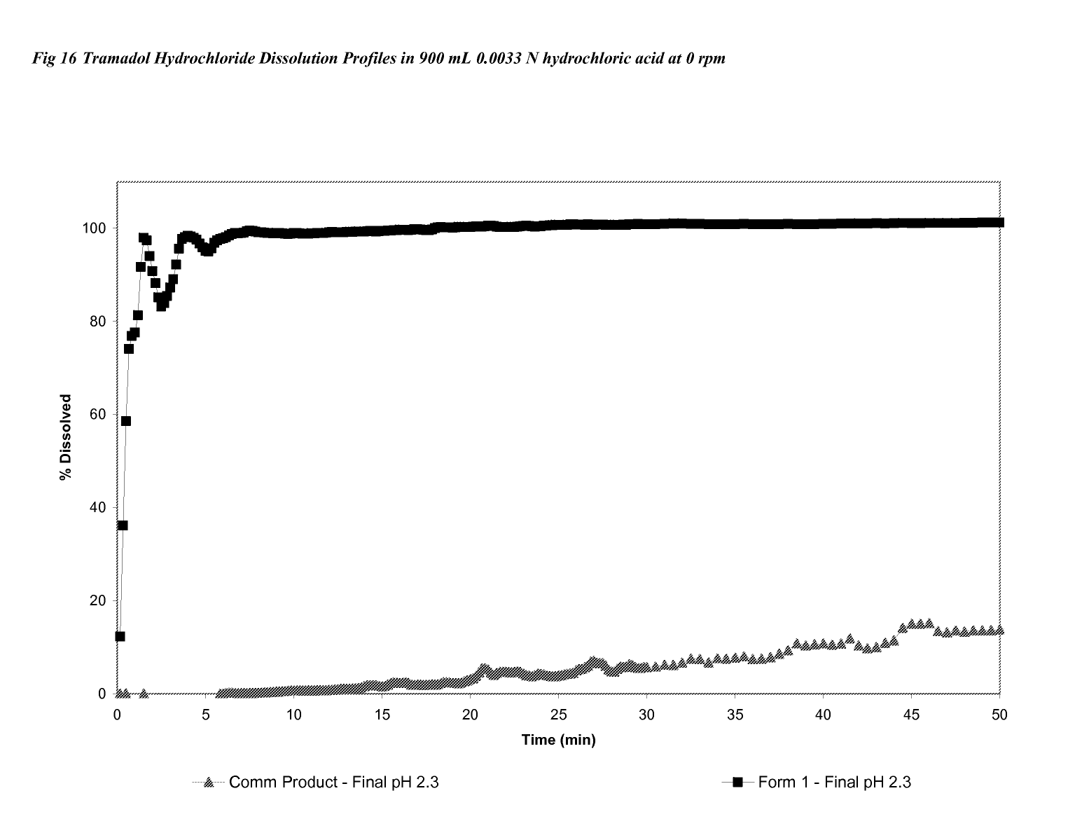

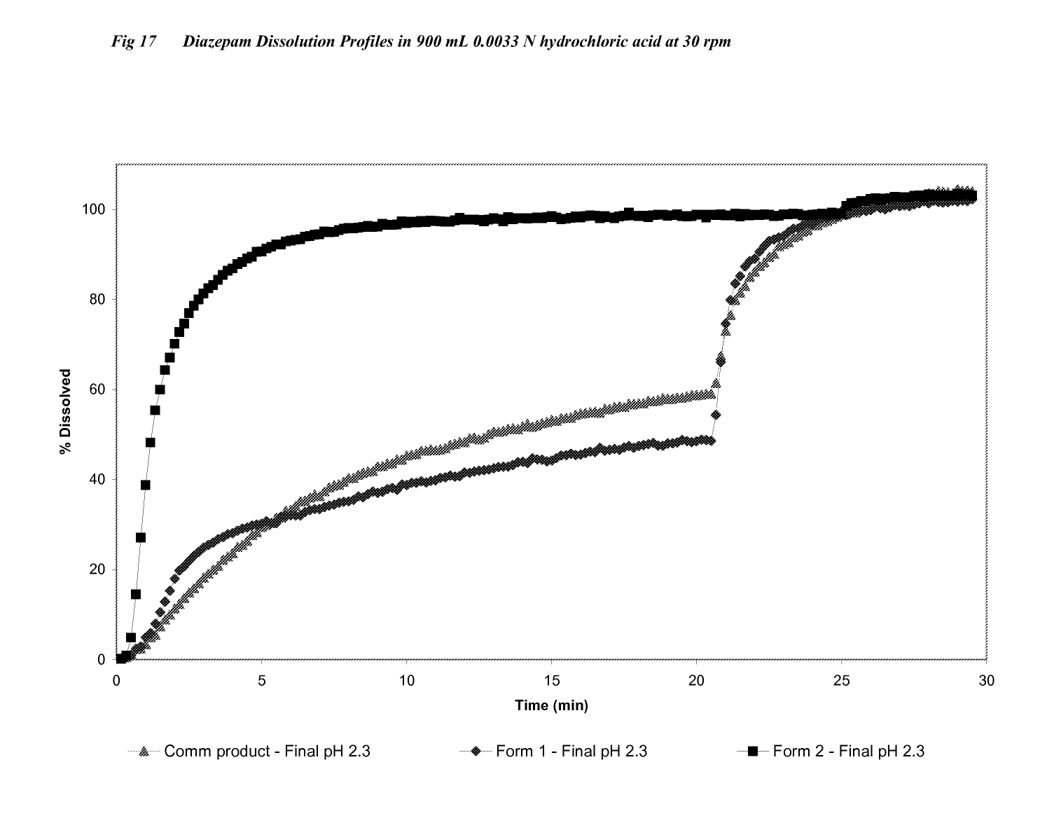

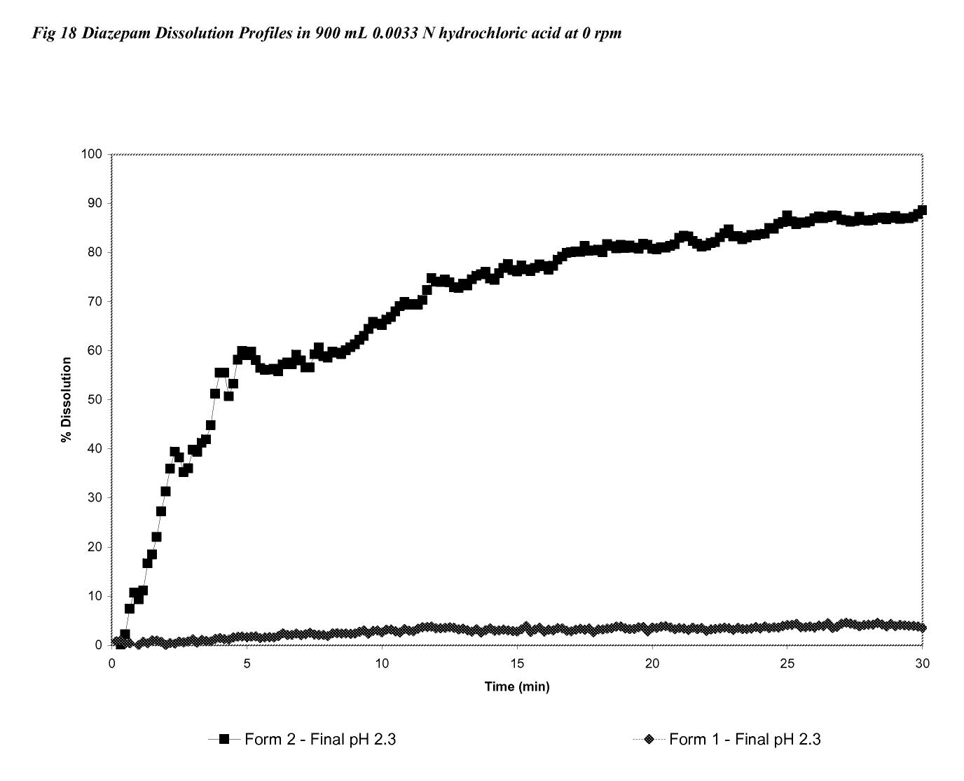

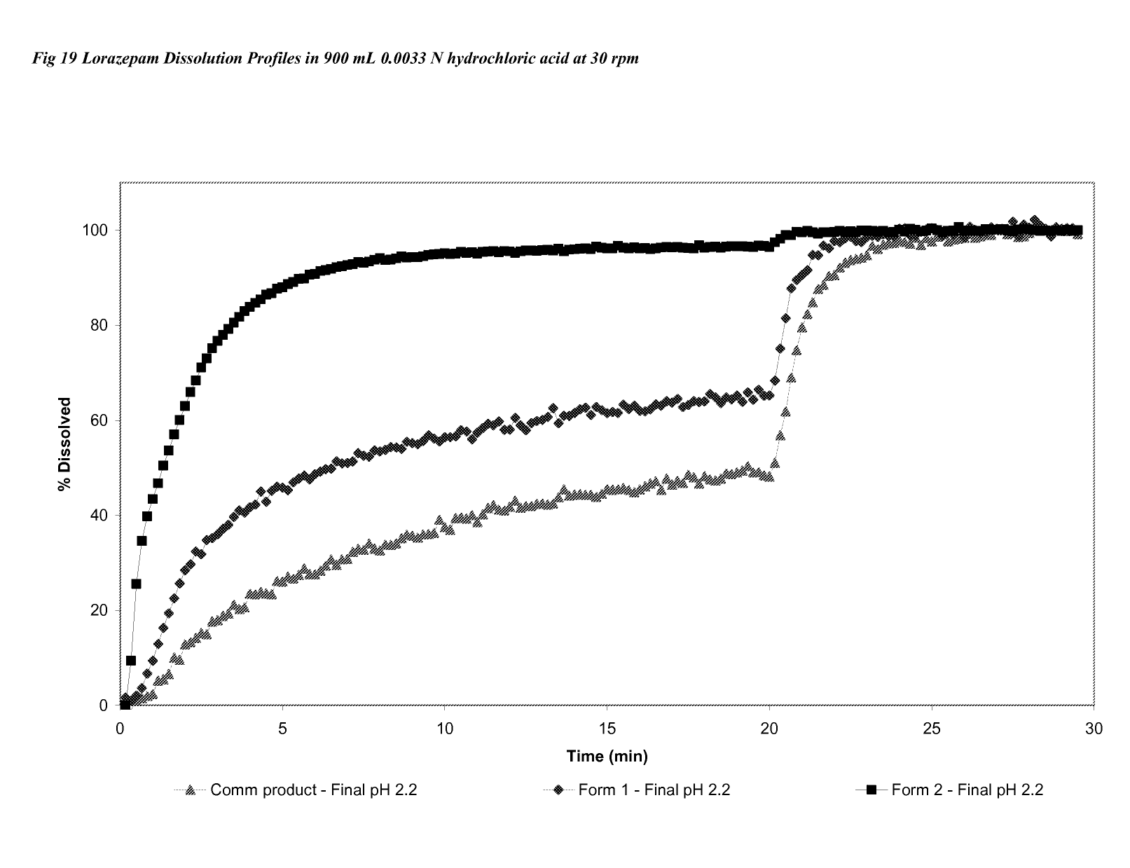

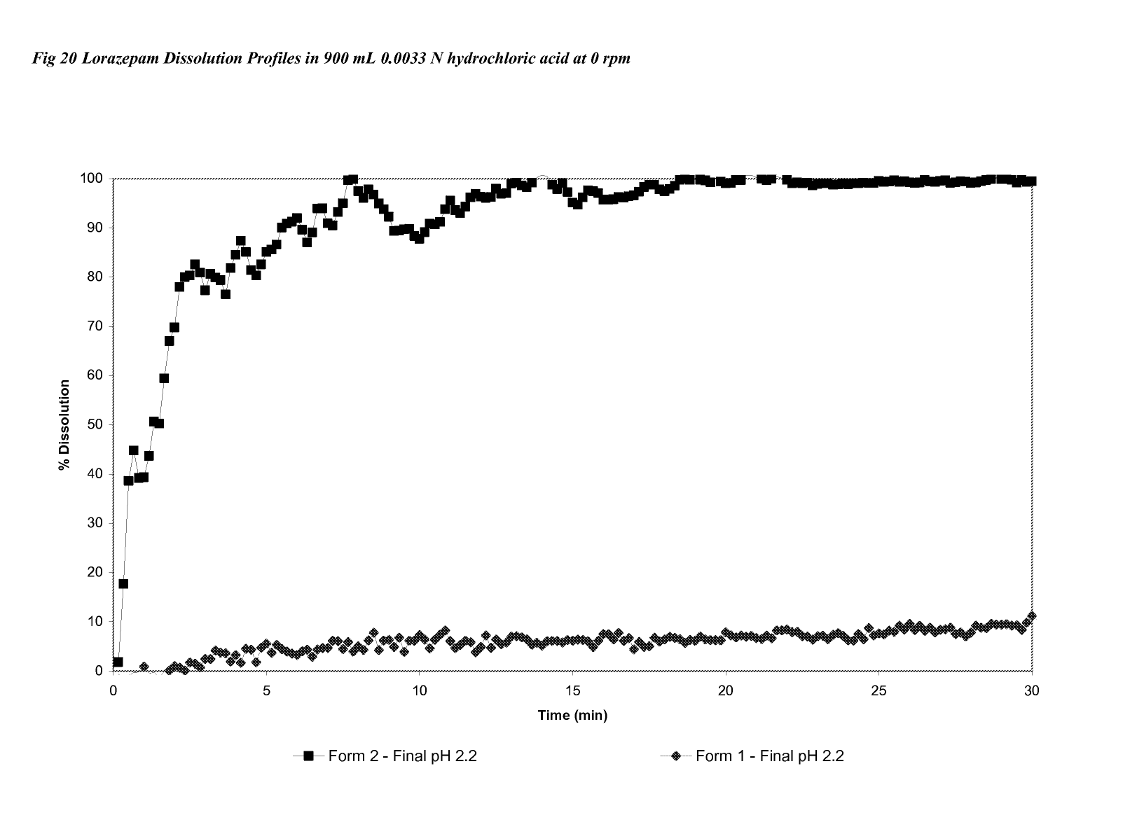

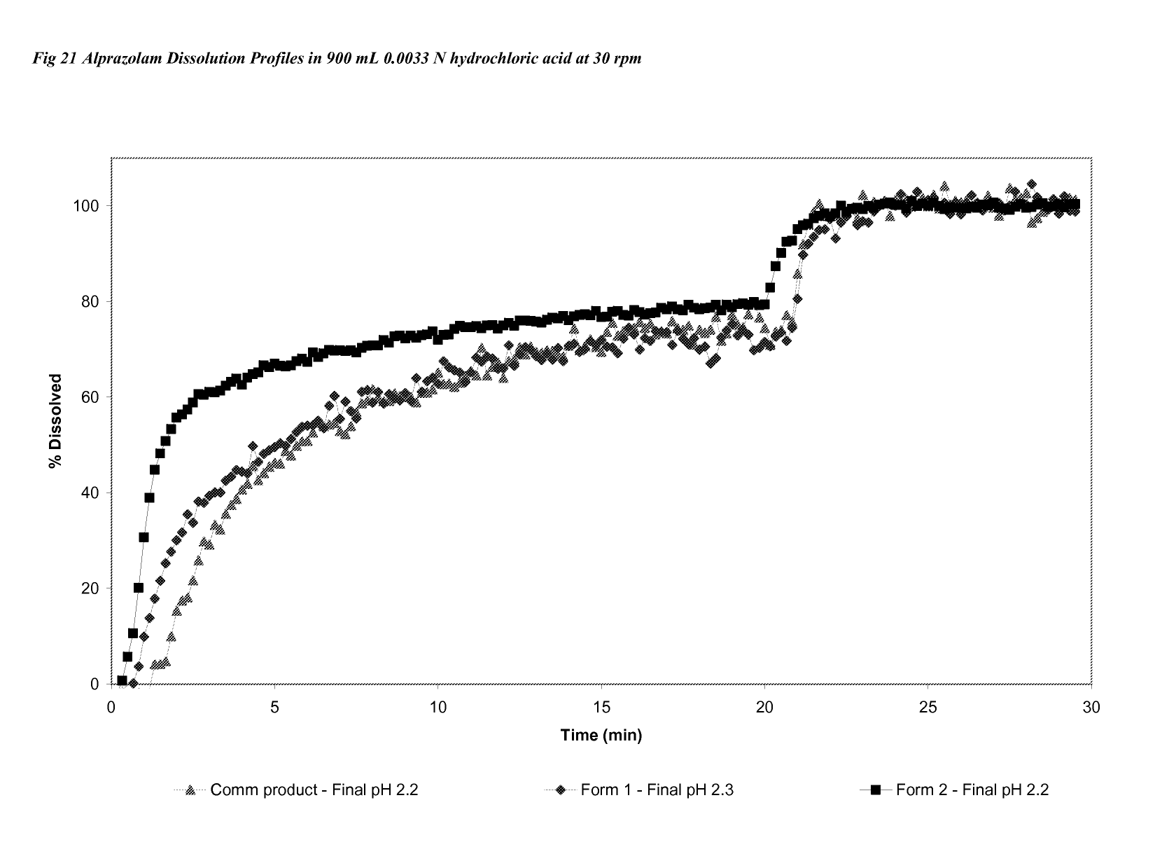

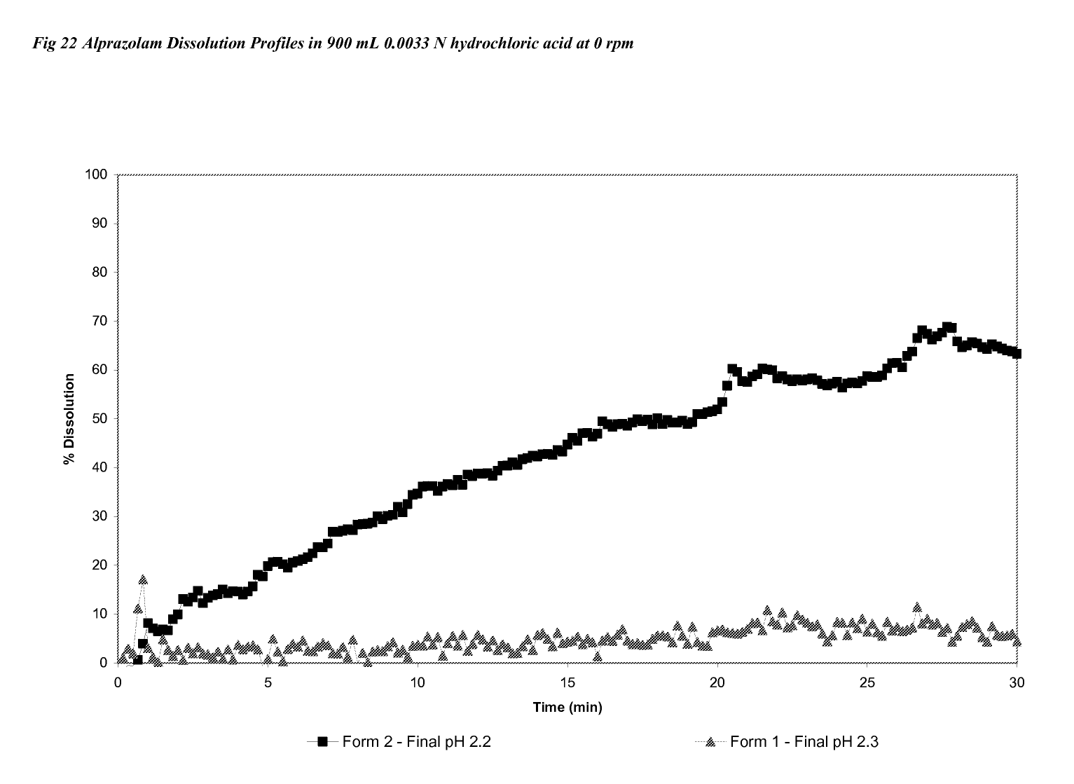

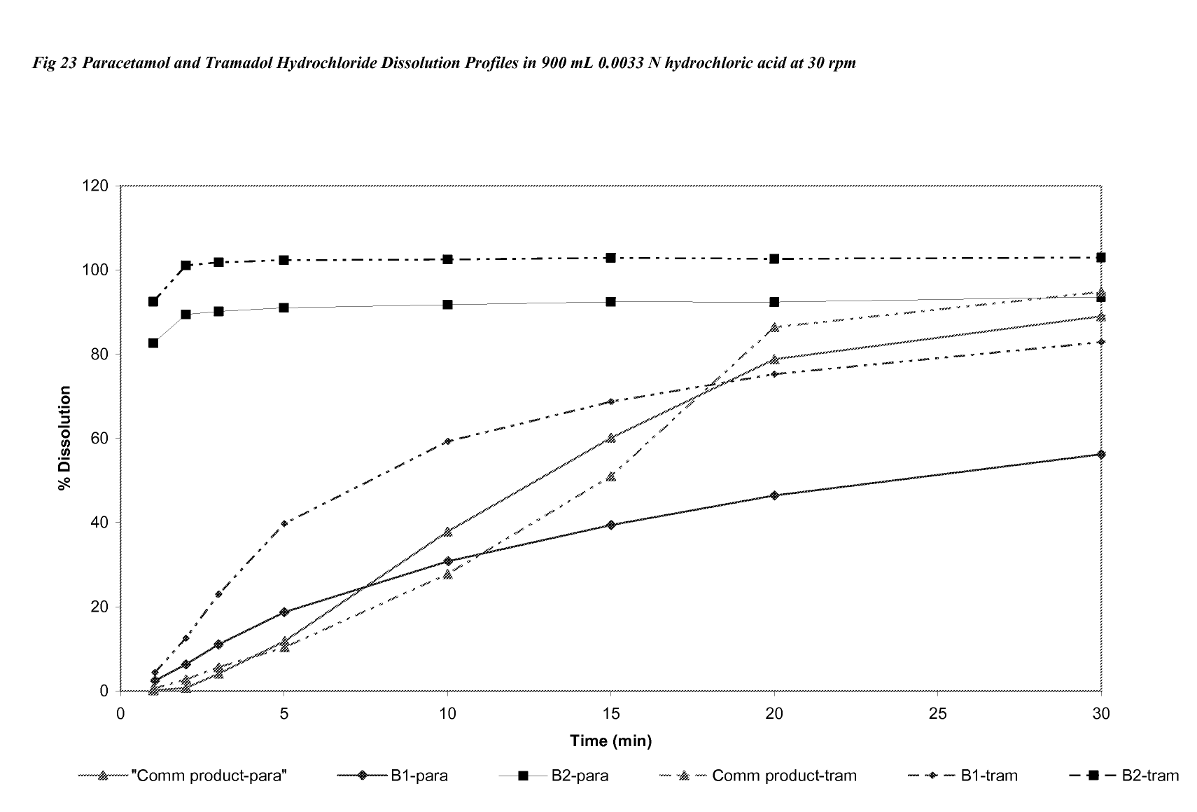

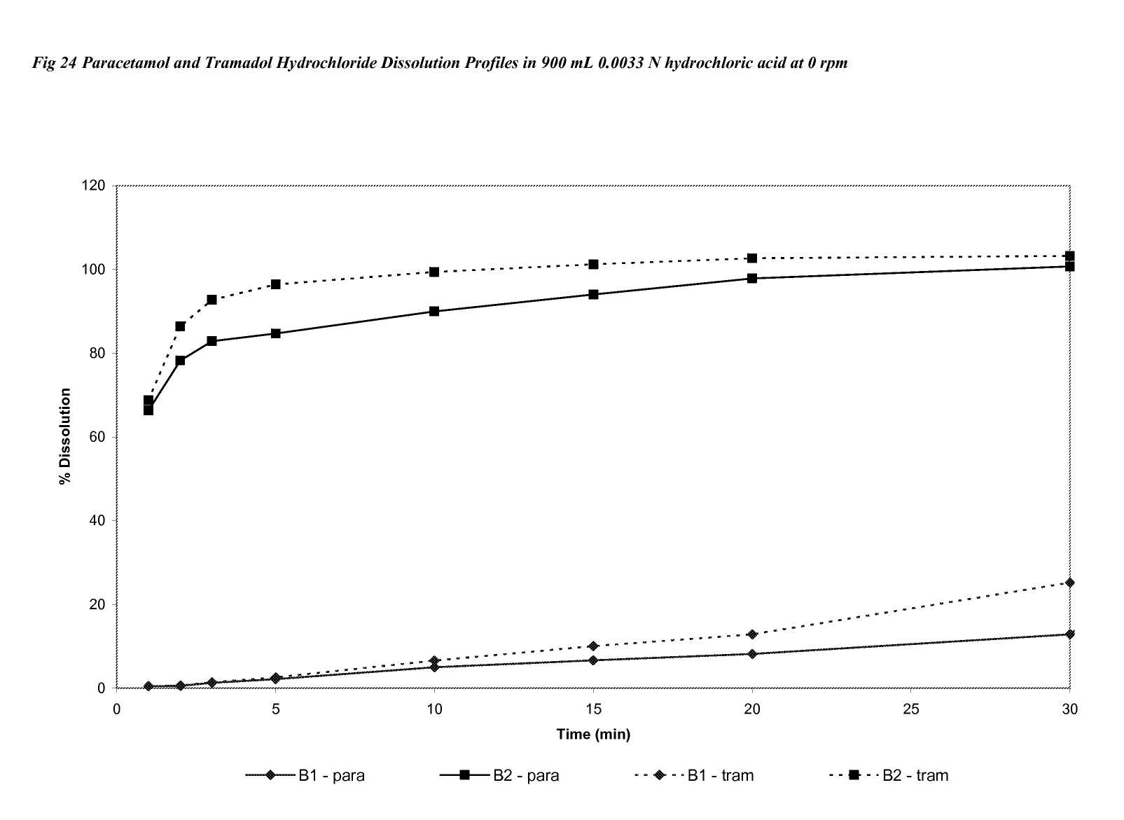

FIGS. 1 to 24 depict graphically the dissolution results for Examples 1 to 11 and 18.

### Conclusions and Further Comments Based on the Examples

It will be apparent that the use of pH modulating agents in accordance with the current invention substantially increases in vitro dissolution of the therapeutic agents exemplified.

Table 1 summarises the dissolution data for formulation examples that demonstrate the current invention in 900 mL 0.0033 N hydrochloric acid using USP apparatus 2 at 30 rpm and 37° C.:

TABLE 1
Dissolution data for basic and amphoteric drugs in formulations
according to the invention at 30 rpm stirring speed in 900 mL
0.0033N hydrochloric acid

| Example | | 120 | 180 | 240 | 300 |
|---|---|---|---|---|---|
| no. | Drug | sec | sec | sec | sec |
| 1 | Fexofenadine hydrochloride | 56 | 63 | 67 | 70 |
| | 180 mg | | | | |
| 2 | Pseudoephedrine | 100 | 101 | 101 | 101 |
| | hydrochloride 60 mg | | | | |
| 3 | Eletriptan hydrobromide 40 mg | 92 | 94 | 95 | 96 |
| 4 | Rizatriptan benzoate 14.53 mg | 93 | 96 | 96 | 96 |
| 5 | Metoclopramide | 79 | 94 | 94 | 95 |
| | hydrochloride 10 mg | | | | |
| 6 | Loperamide hydrochloride 2 mg | 80 | 82 | 83 | 84 |
| 7 | Codeine phosphate 30 mg | 51 | 78 | 89 | 97 |
| 8 | Tramadol hydrochloride | 100 | 100 | 100 | 100 |
| | 37.5 mg | | | | |
| 9 | Diazepam 5 mg | 70 | 81 | 87 | 91 |
| 10 | Lorazepam 2.5 mg | 63 | 77 | 84 | 88 |
| 11 | Alprazolam 1 mg | 56 | 61 | 63 | 67 |

With the exception of fexofenadine hydrochloride and alprazolam, it is clear from these results that the formulations of these drugs containing pH modulating agents according to the invention, had a dissolution rate greater than 70% at 180 seconds at 30 rpm. Fexofenadine hydrochloride and alprazolam did not meet this specification achieving 63% and 61% dissolution respectively at 180 seconds at 30 rpm. However, it should be noted that no formulation optimisation was conducted, and it is expected that better performing examples could be formulated according to the present invention.

Table 2 provides a summary of the dissolution data for all drug formulations exemplified in the specification where the pH modulating agent is a base and an acid, measured in 900 mL 0.0033 N hydrochloric acid using USP apparatus 2 at 0 rpm and 37° C.:

TABLE 2
Summary dissolution data for basic and amphoteric drugs in formulations
according to the invention where the pH modulating agents include a base
and an acid, measured at 0 rpm stirring speed in 900 mL 0.0033N
hydrochloric acid

| Example | | 300 | | 30 |
|---|---|---|---|---|
| no. | Drug | sec | 15 min | min |
| 1 | Fexofenadine hydrochloride 180 mg | 60 | 73 | 81 |
| 2 | Pseudoephedrine hydrochloride | 80 | 100 | 100 |
| | 60 mg | | | |
| 3 | Eletriptan hydrobromide 40 mg | 95 | 98 | 100 |
| 4 | Rizatriptan benzoate 14.53 mg | 102 | 100 | 100 |
| 5 | Metoclopramide hydrochloride | 69 | 86 | 91 |
| | 10 mg | | | |
| 6 | Loperamide hydrochloride 2 mg | 72 | 87 | 95 |
| 7 | Codeine phosphate 30 mg | 75 | 99 | 100 |
| 8 | Tramadol hydrochloride 37.5 mg | 95 | 99 | 100 |
| 9 | Diazepam 5 mg | 59 | 76 | 89 |
| 10 | Lorazepam 2.5 mg | 85 | 95 | 99 |
| 11 | Alprazolam 1 mg | 20 | 45 | 63 |
| 12 | Sildenafil citrate 140 mg | 96 | 95 | 97 |
| 13 | Ondansetron hydrochloride 10 mg | 65 | 79 | 85 |
| 14 | Zolmitriptan 2.5 mg | 77 | 88 | 96 |
| 15 | Zolpidem tartrate 10 mg | 91 | 94 | 96 |
| 16 | Cetirizine hydrochloride 10 mg | 78 | 90 | 96 |

It will be apparent from Table 2 that formulations according to the invention which contain a pharmaceutically acceptable acid in addition to a bicarbonate, demonstrate substantially increased in vitro dissolution of the therapeutic agents exemplified in the absence of external stirring. This highlights the role of intrinsic micro-stirring in enhancing dissolution, where the reaction between the pH modulating agents has a greater effect on dissolution than the reaction between the base and acid in the dissolution medium.

For some drugs, enhanced dissolution was achieved with formulations containing a bicarbonate alone. For others, the addition of an organic acid to the bicarbonate further enhanced the dissolution. In some cases the addition of an acid was necessary to achieve the dissolution performance described in this specification.

In the absence of external stirring, at 0 rpm, the results achieved for formulations containing a base alone are significantly reduced compared to those for formulations containing an acid and a base. This results from the greater intrinsic micro-stirring resulting from the reaction between the base and organic acid relative to the reaction between the base and the acidic dissolution medium.

The extent of dissolution enhancement seen with bicarbonate alone is evident in Table 2a which summarises the dissolution data for drug formulations exemplified in the specification where the pH modulating agent is a base only, measured in 900 mL 0.0033 N hydrochloric acid using USP apparatus 2 at 0 rpm and 37° C. Where the dissolution does not reach adequate levels, acid is added to improve the dissolution to the levels seen in Table 2.

TABLE 2a
Summary dissolution data for basic and amphoteric drugs in formulations
according to the invention where the only pH modulating agent used is a
base, measured at 0 rpm stirring speed in 900 mL 0.0033N
hydrochloric acid

| Example | | 300 | | 30 |
|---|---|---|---|---|
| no. | Drug | sec | 15 min | min |
| 1 | Fexofenadine hydrochloride 180 mg | 49 | 54 | 60 |
| 2 | Pseudoephedrine hydrochloride | 45 | 69 | 102 |
| | 60 mg | | | |
| 5 | Metoclopramide hydrochloride | 5 | 28 | 46 |
| | 10 mg | | | |
| 6 | Loperamide hydrochloride 2 mg | 27 | 43 | 101 |
| 7 | Codeine phosphate 30 mg | 90 | 92 | 96 |
| 9 | Diazepam 5 mg | 1.6 | 2.8 | 3.5 |
| 10 | Lorazepam 2.5 mg | 5.6 | 6.1 | 11.2 |
| 11 | Alprazolam 1 mg | 1 | 4 | 4 |
| 12 | Sildenafil citrate 140 mg | 96 | 95 | 97 |
| 16 | Cetirizine HCl 10 mg | 78 | 90 | 96 |

As seen in Table 2a, in the formulations that contained sodium bicarbonate alone without any additional acid, most drugs achieved more than 5% dissolution in 30 minutes at 0 rpm. While diazepam and alprazolam did not meet this specification at 3.5% and 4% respectively, it should be noted that no formulation optimisation was conducted, and it is expected that better performing examples could be formulated according to the present invention.

### Combination of Drugs

The basic salt tramadol hydrochloride is more soluble than the unionised drug, paracetamol with which it has been formulated, having a solubility in water around 30 mg/mL compared with 14 mg/mL for paracetamol. On a weight for weight basis, the tramadol hydrochloride needs around 1 mL for total dissolution compared to around 27 mL for the dose of paracetamol.

In 900 mL 0.0033 N hydrochloric acid at 30 rpm, only the formulation with base and acid (B2) demonstrates very fast dissolution reaching 100% within 5 minutes.

The formulation with the higher level of bicarbonate alone (28%) shows slower dissolution for both drugs although the dissolution of the more soluble tramadol hydrochloride is faster than that of the paracetamol. Dissolution of this formulation does not show significantly improved dissolution compared with the commercial product.

In 900 mL 0.0033 N hydrochloric acid at 0 rpm, the intrinsic dissolution enhancing features of the formulations become apparent as the effect of the external acidity is reduced and external stirring is eliminated. Under these conditions, formulation B2 containing the base and acid demonstrates fast and significant levels of dissolution compared with the formulation with base alone.

### Conclusion

Based on these results, it is apparent that:
formulations containing bicarbonate alone according to the present invention have a dissolution rate greater than 5% at 30 minutes at 0 rpm, and
formulations containing bicarbonate with a pharmaceutically acceptable acid according to the present invention have a dissolution rate greater than 5% at 300 seconds at 0 rpm.

### Example 1

### A Salt of an Amphoteric Compound

TABLE 3
Fexofenadine Hydrochloride Formulations

| | | | Commercial |
|---|---|---|---|
| Formulation | 1 | 2 | product |
| Fexofenadine hydrochloride (mg) | 180 | 180 | 180 |
| Sodium bicarbonate (mg) | 50 | 50 | 0 |
| Fumaric acid (mg) | 0 | 35 | 0 |
| Microcrystalline cellulose (mg) | ✓+150 | ✓+150 | ✓ |
| Croscarmellose sodium (mg) | ✓+30 | ✓+30 | ✓ |
| Pregelatinised maize starch | ✓ | ✓ | ✓ |
| Magnesium stearate (mg) | ✓ | ✓ | ✓ |
| Total tablet weight (mg) | 850 | 885 | 620 |
| pH modulating agent (%) | 5.9 | 9.6 | 0 |
| Hardness (Kp) | 14 | 14 | >33 |
| Disintegration time in 0.0033M | 60 | <40 | 180 |
| hydrochloric acid (sec) | | | |

Tablets 1 and 2 were compressed using 19 mm×9 mm oval shaped punches.

The commercial product was a 18 mm×8 mm coated oval shaped convex tablet.

TABLE 4
Fexofenadine Hydrochloride Dissolution in 900 mL 0.0033N
hydrochloric acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 46 | 49 | 3 |
| 120 sec | 56 | 55 | 6 |
| 180 sec | 63 | 63 | 13 |
| 5 min | 69 | 69 | 26 |
| 15 min | 76 | 82 | 45 |
| Final pH | 2.4 | 2.4 | 2.3 |

TABLE 5
Fexofenadine Hydrochloride Dissolution in 900 mL 0.0033N hydrochloric
acid at 0 rpm

| | % drug dissolved in | | |
|---|---|---|---|
| | 900 mL 0.0033N | | |
| | hydrochloric acid at 0 rpm | | |
| | | | Commercial |
| Formulation | 1 | 2 | product |
| 90 sec | 30 | 41 | 0 |
| 120 sec | 34 | 46 | 0 |
| 180 sec | 40 | 53 | 0 |
| 5 min | 49 | 60 | 0 |
| 15 min | 54 | 73 | 1 |
| 30 min | 60 | 81 | 4 |
| Final pH | 2.4 | 2.4 | 2.3 |

### Example 2

### A Salt of a Basic Compound

TABLE 6
Pseudoephedrine Hydrochloride Formulations

| | | | Commercial |
|---|---|---|---|
| Formulation | 1 | 2 | product |
| Pseudoephedrine hydrochloride (mg) | 60 | 60 | 60 |
| Sodium bicarbonate (mg) | 30 | 30 | 0 |
| Citric acid anhydrous (mg) | 0 | 23 | 0 |
| Microcrystalline cellulose (mg) | 80 | 120 | 0 |
| Crospovidone (mg) | 15 | 20 | 0 |
| Lactose | ✓ | ✓ | ✓ |
| Magnesium stearate (mg) | 3 | 3 | 0 |
| Total tablet weight (mg) | 365 | 433 | 237 |
| pH modulating agent (%) | 8.2 | 12.2 | 0 |
| Hardness (Kp) | 6 | 3 | 1.5 |
| Disintegration time in 0.0033M | 120 | 40 | 22 |
| hydrochloric acid (Sec) | | | |

Tablets 1 and 2 were compressed using 15 mm×5 mm oval shallow concave punches with a break bar.

The commercial tablets were uncoated 8.5 mm diameter round flat bevelled edge with a break-bar.

TABLE 7
Pseudoephedrine Hydrochloride Dissolution in 900 mL 0.0033N
hydrochloric acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 14 | 87 | 11 |
| 120 sec | 21 | 100 | 16 |
| 180 sec | 31 | 101 | 23 |
| 5 min | 45 | 101 | 35 |
| 15 min | 69 | 102 | 62 |
| Final pH | 2.3 | 2.3 | 2.3 |

TABLE 8
Pseudoephedrine Hydrochloride Dissolution in 900 mL 0.0033N
hydrochloric acid at 0 rpm

% drug
dissolved
in 900 mL
0.0033N
hydrochloric
acid at 0 rpm

| Formulation | 1 | 2 |
|---|---|---|
| 90 sec | 0 | 32 |
| 120 sec | 0 | 34 |
| 180 sec | 2 | 61 |
| 5 min | 4 | 80 |
| 15 min | 17 | 100 |
| 30 min | 33 | 101 |
| Final pH | 2.3 | 2.3 |

### Example 3

### A Salt of a Basic Compound

TABLE 9
Eletriptan Hydrobromide Formulations

| | | | Commercial |
|---|---|---|---|
| Formulation | 1 | 2 | product |
| Eletriptan Hydrobromide (mg) | 48.5 | 48.5 | 48.5 |
| Sodium bicarbonate (mg) | 20 | 40 | 0 |
| Fumaric acid (mg) | 0 | 28 | 0 |
| Microcrystalline cellulose (mg) | ✓+70 | ✓+70 | ✓ |
| Croscarmellose sodium (mg) | ✓+10 | ✓+10 | ✓ |
| Lactose | ✓ | ✓ | ✓ |
| Magnesium stearate (mg) | ✓ | ✓ | ✓ |
| Coating & colouring agents | ✓ | ✓ | ✓ |
| Total tablet weight (mg) | 300 | 348 | 204 |
| pH modulating agent (%) | 6.7 | 19.5 | 0 |
| Hardness (Kp) | 6 | 4 | — |
| Disintegration time in 0.0033M | 28 | 50 | — |
| hydrochloric acid (Sec) | | | |

Tablets 1 and 2 were compressed using 10 mm round shallow concave punches.

The commercial product was coated 8.5 mm diameter round biconvex tablets.

TABLE 10
Eletriptan Hydrobromide Dissolution in 900 mL 0.0033N hydrochloric
acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 27 | 88 | 2 |
| 120 sec | 30 | 92 | 4 |
| 180 sec | 34 | 94 | 9 |
| 5 min | 37 | 96 | 23 |
| 15 min | 48 | 97 | 42 |
| Final pH | 1.7 | 1.7 | 1.8 |

TABLE 11
Eletriptan Hydrobromide Dissolution in 900 mL 0.0033N hydrochloric
acid at 0 rpm

% drug dissolved in
900 mL 0.0033N
hydrochloric acid at 0 rpm

| Formulation | 2 | Commercial product |
|---|---|---|
| 90 sec | 63 | 0 |
| 120 sec | 75 | 0 |
| 180 sec | 88 | 0 |
| 5 min | 95 | 0 |
| 15 min | 98 | 2 |
| 30 min | 100 | 8 |
| Final pH | 2.3 | 2.3 |

### Example 4

### A Salt of a Basic Compound

TABLE 12
Rizatriptan Benzoate Formulations

| | | | Commercial |
|---|---|---|---|
| Formulation | 1 | 2 | product |
| Rizatriptan Benzoate (mg) | 14.53 | 14.53 | 14.53 |
| Sodium bicarbonate (mg) | 10 | 40 | 0 |
| Citric acid anhydrous (mg) | 0 | 30.7 | 0 |
| Microcrystalline cellulose (mg) | 111.97 | 51.27 | ✓ |
| Crospovidone (mg) | 12 | 12 | 0 |
| Lactose Monohydrate | 0 | 0 | ✓ |
| Pregelatinised corn Starch | 0 | 0 | ✓ |
| Ferric oxide | 0 | 0 | ✓ |
| Magnesium stearate (mg) | 1.5 | 1.5 | ✓ |
| Total tablet weight (mg) | 150 | 150 | 194 |
| pH modulating agent (%) | 6.7 | 6.7 | 0 |
| Hardness (Kp) | 5 | 4 | — |
| Disintegration time in 0.0033M | 6 | 32 | — |
| hydrochloric acid (Sec) | | | |

Tablets 1 and 2 were compressed using 8 mm round shallow concave punches.

The commercial product from the USA was an uncoated 12×5 mm oval capsule shaped tablet.

TABLE 13
Rizatriptan Benzoate dissolution in 900 mL 0.0033N hydrochloric
acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 41 | 81 | 28 |
| 120 sec | 45 | 93 | 37 |
| 180 sec | 51 | 96 | 48 |
| 5 min | 58 | 96 | 61 |
| 15 min | 76 | 96 | 77 |
| Final pH | 2.3 | 2.3 | 2.4 |

TABLE 14
Rizatriptan Benzoate dissolution in 900 mL 0.0033N hydrochloric acid
at 0 rpm

| | % drug dissolved in |
|---|---|
| | 900 mL 0.0033N |
| | hydrochloric acid |
| | at 0 rpm |
| Formulation | 2 |
| 90 sec | 80 |
| 120 sec | 91 |
| 180 sec | 94 |
| 5 min | 102 |
| 15 min | 100 |
| 30 min | 100 |
| Final pH | 2.3 |

### Example 5

### A Salt of a Basic Compound

TABLE 15
Metoclopramide Hydrochloride Formulations

| | | | Commercial |
|---|---|---|---|
| Formulation | 1 | 2 | product |
| Metoclopramide Hydrochloride (mg) | 10 | 10 | 10 |
| Sodium bicarbonate (mg) | 20 | 20 | 0 |
| Fumaric acid (mg) | 0 | 15 | 0 |
| Microcrystalline cellulose (mg) | ✓+80 | ✓+80 | ✓ |
| Crospovidone (mg) | 15 | 15 | 0 |
| Lactose anhydrous (mg) | ✓ | ✓ | ✓ |
| Pregelatinised maize starch | ✓3 | ✓3 | ✓ |
| Colloidal anhydrous silica | ✓ | ✓ | ✓ |
| Magnesium stearate (mg) | ✓+3 | ✓+3 | ✓ |
| Total tablet weight (mg) | 244 | 259 | 126 |
| pH modulating agent (%) | 8.2 | 13.5 | 0 |
| Hardness (Kp) | 8 | 8 | — |
| Disintegration time in 0.0033M | 146 | 146 | — |
| hydrochloric acid (Sec) | | | |

Tablets 1 and 2 were compressed using 8 mm round shallow concave punches.

The commercial tablets from the USA were uncoated 7 mm diameter round convex.

TABLE 16
Metoclopramide Hydrochloride dissolution in 900 mL 0.0033N
hydrochloric acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 6 | 49 | 6 |
| 120 sec | 12 | 79 | 8 |
| 180 sec | 22 | 94 | 13 |
| 5 min | 41 | 95 | 24 |
| 15 min | 72 | 97 | 66 |
| Final pH | 2.0 | 2.0 | 2.1 |

TABLE 17
Metoclopramide Hydrochloride dissolution in 900 mL 0.0033N
hydrochloric acid at 0 rpm

% drug
dissolved
in 900 mL
0.0033N
hydrochloric
acid at 0 rpm

| Formulation | 1 | 2 |
|---|---|---|
| 90 sec | 1 | 5 |
| 120 sec | 1 | 9 |
| 180 sec | 2 | 26 |
| 5 min | 5 | 69 |
| 15 min | 28 | 86 |
| 30 min | 46 | 91 |
| Final pH | 2.1 | 2.1 |

### Example 6

### A Salt of a Basic Compound

TABLE 18
Loperamide Hydrochloride Formulations

| | 1 | 2 | Commercial |
|---|---|---|---|
| Formulation | 0522810 | 0522820 | product |
| Loperamide Hydrochloride (mg) | 2 | 2 | 2 |
| Sodium bicarbonate (mg) | 20 | 20 | 0 |
| Malic acid (mg) | 0 | 16 | 0 |
| Microcrystalline cellulose (mg) | ✓+80 | ✓+80 | ✓ |
| Crospovidone (mg) | 15 | 15 | 0 |
| Calcium Phosphate (mg) | ✓ | ✓ | ✓ |
| Colloidal silica anhydrous (mg) | ✓ | ✓ | ✓ |
| Magnesium stearate (mg) | ✓+3 | ✓+3 | ✓ |
| Colour (mg) | ✓ | ✓ | ✓ |
| Total tablet weight (mg) | 268 | 284 | 150 |
| pH modulating agent (%) | 7.5 | 7.5 | 0 |
| Hardness (Kp) | 5.5 | 5 | 5.5 |
| Disintegration time in 0.0033N | 7 | 11 | 57 |
| hydrochloric acid (Sec) | | | |

Tablets 1 and 2 were compressed using 8 mm round shallow concave punches.

The commercial product was an uncoated 9 mm×4.5 mm capsule shaped tablet.

TABLE 19
Loperamide Hydrochloride dissolution in 900 mL 0.0033N hydrochloric
acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 9 | 76 | 10 |
| 120 sec | 12 | 80 | 15 |
| 180 sec | 18 | 82 | 31 |
| 5 min | 27 | 84 | 48 |
| 15 min | 43 | 89 | 79 |
| Final pH | 2.5 | 2.5 | 2.3 |

TABLE 20
Loperamide Hydrochloride Dissolution in 900 mL 0.0033N
hydrochloric acid at 0 rpm

% drug dissolved
in 900 mL 0.0033N
hydrochloric
acid at 0 rpm

| Formulation | 1 | 2 |
|---|---|---|
| 90 sec | 3 | 28 |
| 120 sec | 3 | 50 |
| 180 sec | 7 | 70 |
| 5 min | 12 | 72 |
| 15 min | 20 | 87 |
| 30 min | 26 | 95 |
| Final pH | 2.5 | 2.5 |

### Example 7

### A Salt of a Basic Compound

TABLE 21
Codeine Phosphate Formulations

| | | | Commercial |
|---|---|---|---|
| Formulation | 1 | 2 | product |
| Codeine Phosphate (mg) | 30 | 30 | 30 |
| Sodium bicarbonate (mg) | 20 | 20 | 0 |
| Ascorbic acid (mg) | 0 | 42 | 0 |
| Microcrystalline cellulose (mg) | 80 | 80 | 0 |
| Crospovidone (mg) | 10 | 10 | 0 |
| Gelatin (mg) | ✓ | ✓ | ✓ |
| Maize starch (mg) | ✓ | ✓ | ✓ |
| Propyl hydroxybenzoate (mg) | ✓ | ✓ | ✓ |
| Lactose (mg) | ✓ | ✓ | ✓ |
| Wheat starch | ✓ | ✓ | ✓ |
| Magnesium stearate (mg) | ✓+2 | ✓+2 | ✓ |
| Total tablet weight (mg) | 192 | 234 | 80 |
| pH modulating agent (%) | 10.4 | 26.5 | 0 |
| Hardness (Kp) | 4 | 5 | 4.2 |
| Disintegration time in 0.0033N | 76 | 48 | 310 |
| hydrochloric acid (Sec) | | | |

Tablets 1 and 2 were compressed using 8 mm round shallow concave punches.

The commercial tablets were uncoated 5.6 mm round shallow convex.

TABLE 22
Codeine Phosphate Dissolution in 900 mL 0.0033N
hydrochloric acid at 30 rpm

| | % drug dissolved in | | |
|---|---|---|---|
| | 900 mL 0.0033N | | |
| | hydrochloric acid at 30 rpm | | |
| | | | Commercial |
| Formulation | 1 | 2 | product |
| 90 sec | 30 | 35 | 12 |
| 120 sec | 46 | 51 | 21 |
| 180 sec | 72 | 78 | 34 |
| 5 min | 90 | 97 | 55 |
| 15 min | 92 | 101 | 99 |
| Final pH | 2.4 | 2.4 | 2.3 |

TABLE 23
Codeine Phosphate dissolution in 900 mL 0.0033N
hydrochloric acid at 0 rpm

% drug dissolved in
900 mL 0.0033N
hydrochloric
acid at 0 rpm

| Formulation | 1 | 2 |
|---|---|---|
| 90 sec | 22 | 54 |
| 120 sec | 25 | 52 |
| 180 sec | 28 | 45 |
| 5 min | 42 | 75 |
| 15 min | 78 | 99 |
| 30 min | 92 | 100 |
| Final pH | 2.4 | 2.4 |

### Example 8

### A Salt of a Basic Compound

TABLE 24
Tramadol Hydrochloride Formulations

| | | Commercial |
|---|---|---|
| Formulation | 2 | product |
| Tramadol Hydrochloride (mg) | 37.5 | 50 |
| Sodium bicarbonate (mg) | 40 | 0 |
| Citric acid anhydrous (mg) | 31 | 0 |
| Microcrystalline cellulose (mg) | 79.5 | ✓ |
| Crospovidone (mg) | 10 | 0 |
| Corn starch (mg) | 0 | ✓ |
| Hypromellose (mg) | 0 | ✓ |
| Lactose (mg) | 0 | ✓ |
| Polyethylene glycol (mg) | 0 | ✓ |
| Polysorbate 80 (mg) | 0 | ✓ |
| Sodium starch glycolate (mg) | 0 | ✓ |
| Titanium dioxide (mg) | 0 | ✓ |
| Wax (mg) | 0 | ✓ |
| Magnesium stearate (mg) | 2 | ✓ |
| Total tablet weight (mg) | 200 | 229 |
| pH modulating agent (%) | 35.5 | 0 |
| Hardness (Kp) | 3.5 | — |
| Disintegration time in 0.0033M | 10 | — |
| hydrochloric acid (Sec) | | |

Tablets 2 were compressed using 7 mm round shallow concave punches.

The commercial tablets were coated 13×5 mm capsule shaped.

TABLE 25
Tramadol Hydrochloride dissolution in 900 mL 0.0033N
hydrochloric acid at 30 rpm

% drug dissolved
in 900 mL 0.0033N
hydrochloric acid at
30 rpm

| | | Commercial |
|---|---|---|
| Formulation | 2 | product |
| 90 sec | 100 | 2 |
| 120 sec | 100 | 3 |
| 180 sec | 100 | 6 |
| 5 min | 100 | 15 |
| 15 min | 100 | 56 |
| Final pH | 2.2 | 2.3 |

TABLE 26
Tramadol Hydrochloride Dissolution in 900 mL 0.0033N
hydrochloric acid at 0 rpm

% drug dissolved
in 900 mL 0.0033N
hydrochloric acid
at 0 rpm

| | | Commercial |
|---|---|---|
| Formulation | 2 | product |
| 90 sec | 98 | 0 |
| 120 sec | 91 | 0 |
| 180 sec | 87 | 0 |
| 5 min | 95 | 0 |
| 15 min | 99 | 2 |
| 30 min | 100 | 6 |
| Final pH | 2.3 | 2.3 |

### Example 9

### A Basic Compound

TABLE 27
Diazepam Formulations

| | | | Commercial |
|---|---|---|---|
| Formulation | 1 | 2 | Product |
| Diazepam (mg) | 5 | 5 | 5 |
| Sodium bicarbonate (mg) | 20 | 20 | 0 |
| Fumaric acid (mg) | 0 | 14 | 0 |
| Microcrystalline cellulose (mg) | 80 | 80 | 0 |
| Crospovidone (mg) | 15 | 15 | 0 |
| Maize starch | ✓ | ✓ | ✓ |
| Lactose | ✓ | ✓ | ✓ |
| Colour QY CI147005 (E104) | ✓ | ✓ | ✓ |
| Magnesium stearate (mg) | ✓+3 | ✓+3 | ✓ |
| Total tablet weight (mg) | 288 | 302 | 170 |
| pH modulating agent (%) | 6.94 | 11.2 | 0 |
| Hardness (Kp) | 10 | 8 | — |
| Disintegration time in 0.0033M | 79 | 37 | — |
| hydrochloric acid (Sec) | | | |

Tablets 1 and 2 were compressed using 8 mm round shallow concave punches.

The commercial tablets were uncoated 8 mm round flat bevelled edge with break bar.

TABLE 28
diazepam Dissolution in 900 mL 0.0033N hydrochloric acid at 30 rpm

| | % drug dissolved in | | |
|---|---|---|---|
| | 900 mL 0.0033N | | |
| | hydrochloric acid | | |
| | at 30 rpm | | |
| | | | Commercial |
| Formulation | 1 | 2 | Product |
| 90 sec | 10 | 60 | 7 |
| 120 sec | 18 | 70 | 11 |
| 180 sec | 25 | 81 | 18 |
| 240 sec | 28 | 87 | 24 |
| 5 min | 30 | 91 | 29 |
| 15 min | 44 | 98 | 53 |
| Final pH | 2.3 | 2.3 | 2.3 |

TABLE 29
Diazepam dissolution in 900 mL 0.0033N hydrochloric acid at 0 rpm

% drug dissolved
in 900 mL 0.0033N
hydrochloric acid
at 0 rpm

| Formulation | 1 | 2 |
|---|---|---|
| 90 sec | 1 | 19 |
| 120 sec | 0 | 31 |
| 180 sec | 1 | 40 |
| 5 min | 2 | 59 |
| 15 min | 3 | 76 |
| 30 min | 4 | 89 |
| Final pH | 2.3 | 2.3 |

### Example 10

### An Amphoteric Compound

TABLE 30
Lorazepam Formulations

| | | | Commercial |
|---|---|---|---|
| Formulation | 1 | 2 | Product |
| Lorazepam (mg) | 2.5 | 2.5 | 2.5 |
| Sodium bicarbonate (mg) | 40 | 40 | 0 |
| Fumaric acid (mg) | 0 | 28 | 0 |
| Microcrystalline cellulose (mg) | 100 | 100 | 0 |
| Crospovidone (mg) | 25 | 25 | 0 |
| Lactose & other excipients | ✓ | ✓ | ✓ |
| Magnesium stearate (mg) | 2 | 2 | 0 |
| Total tablet weight (mg) | 365 | 393 | 198 |
| pH modulating agent (%) | 11 | 17.3 | 0 |
| Hardness (Kp) | 10 | 9 | — |
| Disintegration time in 0.0033M | 10 | 10 | 6 |
| hydrochloric acid (Sec) | | | |

Tablets 1 and 2 were compressed using 15 mm×5 mm oval shallow concave punches with break bar.

The commercial tablets were uncoated 7 mm round convex with an enlarged break bar.

TABLE 31
Lorazepam dissolution in 900 mL 0.0033N hydrochloric acid at 30 rpm

| | % drug dissolved in | | |
|---|---|---|---|
| | 900 mL 0.0033N | | |
| | hydrochloric acid at | | |
| | 30 rpm | | |
| | | | Commercial |
| Formulation | 1 | 2 | Product |
| 90 sec | 19 | 54 | 7 |
| 120 sec | 28 | 63 | 13 |
| 180 sec | 36 | 77 | 18 |
| 240 sec | 42 | 84 | 24 |
| 5 min | 46 | 88 | 26 |
| 30 min | 100 | 100 | 99 |
| Final pH | 2.2 | 2.2 | 2.2 |

TABLE 32
Lorazepam dissolution in 900 mL 0.0033N hydrochloric acid at 0 rpm

% drug dissolved
in 900 mL 0.0033N
hydrochloric acid
at 0 rpm

| Formulation | 1 | 2 |
|---|---|---|
| 90 sec | 0 | 50 |
| 120 sec | 1 | 70 |
| 180 sec | 2 | 77 |
| 5 min | 6 | 85 |
| 15 min | 6 | 95 |
| 30 min | 11 | 99 |
| Final pH | 2.2 | 2.2 |

### Example 11

### A Basic Compound

TABLE 33
Alprazolam Formulations

| Formulation | 1 | 2 | Commercial Product |
|---|---|---|---|
| Alprazolam (mg) | 1 | 1 | 1 |
| Sodium bicarbonate (mg) | 20 | 20 | 0 |
| Fumaric acid (mg) | 0 | 14 | 0 |
| Microcrystalline cellulose (mg) | ✓+80 | ✓+80 | ✓ |
| Crospovidone (mg) | 15 | 15 | 0 |
| Lactose | ✓ | ✓ | ✓ |
| Maize starch | ✓ | ✓ | ✓ |
| Sodium benzoate | ✓ | ✓ | ✓ |
| Docusate sodium | ✓ | ✓ | ✓ |
| Povidone | ✓ | ✓ | ✓ |
| Colloidal anhydrous silica | ✓ | ✓ | ✓ |
| Sodium starch glycolate | ✓ | ✓ | ✓ |
| Magnesium stearate (mg) | ✓+3 | ✓+3 | ✓ |
| Indigo carmine CI 73015 | ✓ | ✓ | ✓ |
| Total tablet weight (mg) | 248 | 262 | 130 |
| pH modulating agent (%) | 8.1 | 13.0 | 0 |
| Hardness (Kp) | 9 | 8 | — |
| Disintegration time in 0.0033M | 74 | 67 | 180 |
| hydrochloric acid (Sec) | | | |

Tablets 1 and 2 were compressed using 8 mm round shallow concave punches.

The commercial product was a flat 9 mm×5 mm oval uncoated tablet with a break-bar.

TABLE 34
Alprazolam dissolution in 900 mL 0.0033N hydrochloric
acid at 30 rpm

| | % drug dissolved | | |
|---|---|---|---|
| | in 900 mL 0.0033N | | |
| | hydrochloric acid | | |
| | at 30 rpm | | |
| | | | Commercial |
| Formulation | 1 | 2 | product |
| 90 sec | 22 | 48 | 4 |
| 120 sec | 30 | 56 | 15 |
| 180 sec | 39 | 61 | 29 |
| 5 min | 50 | 67 | 46 |
| Final pH | 2.2 | 2.3 | 2.2 |

TABLE 35
Alprazolam dissolution in 900 mL 0.0033N hydrochloric acid at 0 rpm

% drug dissolved in 900 mL 0.0033N
hydrochloric acid at 0 rpm

| Formulation | 1 | 2 |
|---|---|---|
| 90 sec | 0 | 7 |
| 120 sec | 0 | 10 |
| 180 sec | 1 | 13 |
| 5 min | 1 | 20 |
| 15 min | 4 | 45 |
| 30 min | 4 | 63 |
| Final pH | 2.2 | 2.2 |

### Example 12

### A Salt of a Basic Compound

TABLE 36
Sildenafil Citrate Formulations

| | Formulation | | | |
|---|---|---|---|---|
| | 1 | 2 | 3 | 4 |
| Sodium bicarbonate (mg) | 0 | 50 | 0 | 50 |
| Potassium bicarbonate (mg) | 0 | 0 | 50 | 0 |
| Microcrystalline cellulose (mg) | 370 | 320 | 280 | 255 |
| Croscarmellose sodium (mg) | 25 | 25 | 25 | 35 |
| Sildenafil citrate(mg) | 100 | 100 | 140 | 140 |
| Magnesium stearate (mg) | 5 | 5 | 5 | 5 |
| Providone K-30 (mg) | 0 | 0 | 0 | 4.4 |
| Carbonate (%) | 0 | 10 | 10 | 10.2 |
| Total (mg) | 500 | 500 | 500 | 489.4 |

TABLE 37
Formulation 4 for a Sildenafil Citrate Granulation

| No | Ingredient | mg/tablet |
|---|---|---|
| Part 1 | | |
| 1 | Sildenafil citrate | 140 |
| 2 | Microcrystalline cellulose | 205 |
| 3 | Croscarmellose sodium | 20 |
| 4 | Providone K-30 (PVP) | 4.4 |
| 5 | Water | — |
| Part 2 | | |
| 5 | Sodium bicarbonate | 50 |
| 6 | Croscarmellose sodium | 15 |
| 7 | Microcrystalline cellulose | 50 |
| 8 | Magnesium stearate | 5 |

### Procedure

### Part 1

A. Prepare a 1.3% w/w solution of 4 in 5.
B. Blend 1, 2 and 3.
C. Spray A onto B in a granulator or mixer to produce a granule suitable for compression
D. Dry item C at 50° C. to a moisture content ˜3%
E. Screen item D through a 850 micron sieve.
F. Screen ingredients 5-7 through a 250 micron sieve.
G. Blend Part 1 with F.
H. Screen ingredient 8 through a 250 micron sieve.
I. Blend G with H
J. Compress.

TABLE 38
Sidenafil Citrate dissolution data in 900 mL
0.0033N hydrochloric acid at 30 rpm

| | % drug dissolved in 900 mL | | |
|---|---|---|---|
| | 0.0033N hydrochloric acid at 30 rpm | | |
| Formulation | 90 sec | 120 sec | 180 sec |
| 1 | 13.2 | 15.2 | 18.2 |
| 2 | >99 | >99 | >99 |
| 3 | 89.5 | 93.5 | 97.3 |
| 4 | 94.4 | 97.5 | >99 |

### Example 13

### A Salt of a Basic Compound

TABLE 39
Ondansetron Hydrochloride Formulations

Formulation

| | 1 | 2 |
|---|---|---|
| Sodium bicarbonate (mg) | 0 | 20 |
| Microcrystalline cellulose | 180 | 140 |
| (mg) | | |
| Crospovidone (mg) | 10 | 10 |
| Glycine (mg) | 0 | 18 |
| Ondansetron hydrochloride | 8 | 10 |
| (mg) | | |
| Magnesium stearate (mg) | 2 | 2 |
| Carbonate (%) | 0 | 10 |
| Total (mg) | 200 | 200 |

TABLE 40
Ondansetron Hydrochloride dissolution data in
900 mL 0.0033N hydrochloric acid at 30 rpm

| | % drug dissolved in 900 mL | | |
|---|---|---|---|
| | 0.0033N hydrochloric acid at 30 rpm | | |
| Formulation | 90 sec | 120 sec | 180 sec |
| 1 | 36.5 | 44.7 | 55.3 |
| 2 | 73.0 | 80.6 | 87.9 |

### Example 14

### A Basic Compound

TABLE 41
Zolmitriptan Formulations

Formulation

| | 1 | 2 |
|---|---|---|
| Sodium bicarbonate (mg) | 0 | 50 |
| Microcrystal cellulose (mg) | 110.3 | 97.1 |
| Sodium starch glycolate (mg) | 6 | 10 |
| Citric acid anhydrous (mg) | 0 | 38.4 |
| Zolmitriptan (mg) | 2.5 | 2.5 |
| Magnesium stearate (mg) | 1.2 | 2 |
| Carbonate (%) | 0 | 25 |
| Total (mg) | 120 | 200 |

TABLE 42
Zolmitriptan dissolution data in 900 mL
0.0033N hydrochloric acid at 30 rpm

| | % drug dissolved in 900 mL | | |
|---|---|---|---|
| | 0.0033N hydrochloric acid at 30 rpm | | |
| Formulation | 90 sec | 120 sec | 180 sec |
| 1 | 35.5 | 42.3 | 51.7 |
| 2 | 95.1 | 97.3 | 98.9 |

### Example 15

### A Salt of a Basic Compound

TABLE 43
Zolpidem Tartrate Formulations

| | Formulation | | |
|---|---|---|---|
| | 1 | 2 | 3 |
| Zolpidem tartrate (mg) | 10 | 10 | 10 |
| Sodium bicarbonate (mg) | 0 | 50 | 50 |
| Microcrystalline cellulose | 178 | 83 | 89.6 |
| (mg) | | | |
| Sodium starch glycolate (mg) | 10 | 10 | 10 |
| Tartaric acid 99% (mg) | 0 | 45 | 0 |
| Citric acid anhydrous (mg) | 0 | 0 | 38.4 |
| Magnesium stearate (mg) | 2 | 2 | 2 |
| Carbonate (%) | 0 | 25 | 25 |
| Total (mg) | 200 | 200 | 200 |

TABLE 44
Zolpidem Tartrate dissolution data in 900 mL
0.0033N hydrochloric acid at 30 rpm

| | % drug dissolved in 900 mL | | |
|---|---|---|---|
| | 0.0033N hydrochloric acid at 30 rpm | | |
| Formulation | 90 sec | 120 sec | 108 sec |
| 1 | 49.4 | 55.8 | 62.3²⁰ |
| 2 | 89.4 | 91.8 | 92.6 |
| 3 | 94.4 | 35.9 | 96.1 |

### Example 16

### A Salt of an Amphoteric Compound

TABLE 45
Cetirizine Dihydrochoride Formulations

| | Formulations | | |
|---|---|---|---|
| | 1 | 2 | 3 |
| Cetirizine dihydrochloride (mg) | 10 | 10 | 10 |
| Sodium bicarbonate (mg) | 0 | 20 | 6 |
| Microcrystalline cellulose (mg) | 178 | 158 | 172 |
| Crospovidone (mg) | 10 | 10 | 10 |
| Magnesium stearate (mg) | 2 | 2 | 2 |
| Carbonate (%) | 0 | 10 | 3 |
| Total (mg) | 200 | 200 | 200 |

TABLE 46
Cetirizine Dihydrochloride dissolution data in 900 mL
0.0033N hydrochloric acid at 30 rpm

| | % drug dissolved in 900 mL | | |
|---|---|---|---|
| | 0.0033N hydrochloric acid at 30 rpm | | |
| Formulation | 90 sec | 120 sec | 180 sec |
| 1 | 27.7 | 36.8 | 47.8 |
| 2 | 73.9 | 79 | 84.1 |
| 3 | 75.5 | 79.6 | 82.9 |

### Example 17

### Comparative Tables of Results for Examples 1 to 16

TABLE 47
Dissolution profiles for basic and amphoteric drugs in formulations
according to the invention at 0 and 30 rpm stirring speed in 900 mL 0.0033N
hydrochloric acid

| | | pH | | % dissolved in 900 mL 0.0033N HCl | | | | | | | |
|---|---|---|---|---|---|---|---|---|---|---|---|
| | | modulating | | Time | | | | | | | |
| | | agent | | 180 sec | | 300 sec | | 15 minutes | | 30 minutes | |
| | | Base | Acid | Stirring speed | | | | | | | |
| Ex. no | Drug | mg/tab | | 0 rpm | 30 rpm | 0 rpm | 30 rpm | 0 rpm | 30 rpm | 0 rpm | 30 rpm |
| 1 | Fexofenadine | 50 | 35 | 53 | 63 | 60 | 69 | 73 | 82 | 81 | 97 |
| | hydrochloride | | | | | | | | | | |
| 2 | Pseudoephedrine | 30 | 23 | 61 | 101 | 80 | 101 | 100 | 101 | 100 | 101 |
| | hydrochloride | | | | | | | | | | |
| 3 | Eletriptan | 40 | 28 | 88 | 94 | 95 | 96 | 98 | 97 | 100 | 99 |
| | hydrochloride | | | | | | | | | | |
| 4 | Rizatriptan | 50 | 38.4 | 94 | 96 | 102 | 96 | 100 | 96 | 100 | 97 |
| | benzoate | | | | | | | | | | |
| 5 | Metoclopramide | 20 | 15 | 26 | 94 | 69 | 95 | 86 | 97 | 91 | 100 |
| | hydrochloride | | | | | | | | | | |
| 6 | Loperamide | 20 | 16 | 70 | 82 | 72 | 84 | 87 | 89 | 95 | 100 |
| | hydrochloride | | | | | | | | | | |
| 7 | Codeine | 20 | 42 | 45 | 78 | 75 | 97 | 99 | 101 | 100 | 101 |
| | phosphate | | | | | | | | | | |
| 8 | Tramadol | 40 | 31 | 87 | 100 | 95 | 100 | 99 | 100 | 100 | 100 |
| | hydrochloride | | | | | | | | | | |
| 9 | Diazepam | 20 | 14 | 40 | 81 | 59 | 91 | 76 | 98 | 89 | 103 |
| 10 | Lorazepam | 40 | 28 | 77 | 77 | 85 | 88 | 95 | 96 | 99 | 100 |
| 11 | Alprazolam | 20 | 14 | 13 | 61 | 20 | 67 | 45 | 77 | 63 | 100 |
| 12 | Sildenafil citrate | 50 | — | 92 | 97 | 96 | 100 | 95 | 100 | 97 | 100 |
| 13 | Ondansetron | 20 | 18 | 50 | 88 | 65 | 96 | 79 | 100 | 85 | 100 |
| | hydrochloride | | | | | | | | | | |
| 14 | Zolmitriptan | 50 | 38.4 | 66 | 73 | 77 | 76 | 88 | 86 | 96 | 98 |
| 15 | Zolpidem tartrate | 50 | 38 | 77 | 96 | 91 | 96 | 94 | 97 | 96 | 98 |
| 16 | Cetirizine | 6 | — | 67 | 84 | 78 | 87 | 90 | 92 | 96 | 96 |
| | dihydrochloride | | | | | | | | | | |

TABLE 48
Solubility Data for a range of basic and amphoteric drugs and
salts thereof which demonstrate enhanced dissolution when
formulated according to the invention

| | | | | Volume of | |
|---|---|---|---|---|---|
| | | | Solubility in | water to | |
| | | Dose | water | dissolve dose | |
| Ex. no. | Drug | (mg) | (mg/mL) | (mL) | Type |
| 1 | Fexofenadine | 60 | 0.8 | 75 | Base salt |
| | hydrochloride | | | | |
| 2 | Pseudoephedrine | 60 | 2000 | 0.03 | Base salt |
| | hydrochloride | | | | |
| 3 | Eletriptan hydrobromide | 48 | 4 | 4 | Base salt |
| 4 | Rizatriptan benzoate | 14 | 42* | 0.3 | Base salt |
| 5 | Metoclopramide | 10 | 0.2* | 50 | Base salt |
| | hydrochloride | | | | |
| 6 | Loperamide | 2 | 0.08 | 2 | Base salt |
| | hydrochloride | | | | |
| 7 | Codeine phosphate | 30 | 435 | 0.07 | Base salt |
| 8 | Tramadol hydrochloride | 37.5 | 30 | 1.3 | Base salt |
| 9 | Diazepam | 5 | 0.04 | 125 | Base |
| 10 | Lorazepam | 2.5 | 0.08 | 31 | Amphoteric |
| 11 | Alprazolam | 1 | 0.07 | 14 | Base |
| 12 | Sildenafil citrate | 140 | 3.5 | 40 | Base salt |
| 13 | Ondansetron | 10 | 2.42 | 4 | Base salt |
| | hydrochloride | | | | |
| 14 | Zolmitriptan | 2.5 | 1.3 | 2 | Base |
| 15 | Zolpidem tartrate | 10 | 23 | 0.4 | Base salt |
| 16 | Cetirizine | 10 | 0.1* | 100 | Amphoteric |
| | dihydrochloride | | | | salt |

*solubility of the base not salt

### Example 18

### Paracetamol and Tramadol Hydrochloride

TABLE 49
Paracetamol and Tramadol Hydrochloride Formulations

| | | | B2 |
|---|---|---|---|
| Item | Formulation | B1 (base alone) | (base + acid) |
| 1 | Paracetamol (mg) | 325 | 162.5 |
| 2 | Sodium bicarbonate (mg) | 200 | 100 |
| 3 | Crospovidone (mg) | 10 | 12.5 |
| 4 | Povidone (mg) | 0 | 8.4 |
| 5 | Water | 0 | 50 |
| 6 | Paracetamol (mg) | 0 | 162.5 |
| 7 | Tramadol Hydrochloride (mg) | 37.5 | 37.5 |
| 8 | Fumaric acid (mg) | 0 | 34 |
| 9 | Microcrystalline cellulose (mg) | 0 | 50 |
| 10 | Crospovidone (mg) | 0 | 12.5 |
| 11 | Povidone (mg) | 13.67 | 5 |
| 12 | Water | 55 | 33 |
| 13 | Prosolve (mg) | 50 | 0 |
| 14 | Microcrystalline cellulose (mg) | 0 | 50 |
| 15 | Crospovidone (mg) | 60 | 60 |
| 16 | Magnesium stearate (mg) | 0 | 7 |
| 17 | Steric acid (mg) | 8 | 0 |

| | | |
|---|---|---|
| Total tablet weight (mg) | 704.17 | 701.9 |
| pH modulating agent (%) | 28.4 | 19.1 |
| Hardness (Kp) | 14 | 10 |
| Disintegration Time in 0.05N hydrochloric | 16 | 20 |
| acid (Sec) | | |

### Method for Formulation 1

### Part 1

A. Prepare a solution of 11 in 12.
B. Blend items 1, 3, and 7.
C. Spray A onto B in a granulator or mixer to form a granule suitable for compression.
D. Dry at 50° C. to achieve moisture content <1%.

### Part 2

E. Screen granules from D through a 1,000 μm sieve.
F. Screen items 2, 13 and 15 through a 280 μm sieve.
G. Blend E with F.
H. Screen item 17 through a 280 μm sieve.
I. Blend G with H.
J. Compress using 19 mm×7 mm oval shallow concave tooling with a break bar on one face to suitable hardness and disintegration time.

### Method for Formulation 2

### Part 1

A. Prepare a solution of 11 in 12.
B. Blend items 6, 7, 8, 9 and 10.
C. Spray A onto B in a granulator or mixer to form a granule suitable for compression.
D. Dry at 70° C. inlet temperature in a fluid bed dryer to a loss on drying of ˜1% after heating at 50° C. for 20 minutes.

### Part 2

E. Prepare a solution of 4 in 5.
F. Blend items 1, 2 and 3.
G. Spray A onto B in a granulator or mixer to form a granule suitable for compression.
H. Dry at 70° C. inlet temperature in a fluid bed dryer to a loss on drying of <1% after heating at 50° C. for 20 minutes.

### Part 3

I. Screen granules from steps D & H through a 500 μm sieve.
J. Screen items 14 and 15 through a 280 μm sieve.
K. Blend Part I with J.
L. Screen item 16 through a 280 μm sieve.
M. Blend K with L.
N. Compress using 19 mm×7 mm oval shallow concave tooling with a break bar on one face to suitable hardness and disintegration time.

TABLE 50
Paracetamol and Tramadol Hydrochloride dissolution in
900 mL 0.0033N hydrochloric acid at 30 rpm

| | % drug dissolved in 900 mL 0.0033N | | | | | |
|---|---|---|---|---|---|---|
| | hydrochloric acid at 30 rpm | | | | | |
| | | | | | Commercial | |
| | B1 (base alone) | | B2 (base + acid) | | Product | |
| Formu- | Parace- | | Parace- | | Parace- | |
| lation | tamol | Tramadol | tamol | Tramadol | tamol | Tramadol |
| 120 sec | 6 | 13 | 89 | 101 | 1 | 3 |
| 180 sec | 11 | 23 | 90 | 102 | 4 | 6 |
| 300 sec | 19 | 40 | 91 | 102 | 12 | 10 |
| 15 min | 39 | 69 | 92 | 103 | 60 | 51 |
| 30 min | 56 | 83 | 94 | 103 | 89 | 95 |

| | | | |
|---|---|---|---|
| Final | 2.5 | 2.4 | 2.2 |
| pH | | | |

TABLE 51
Paracetamol and Tramadol Hydrochloride dissolution in
900 mL 0.0033N hydrochloric acid at 0 rpm

| | % drug dissolved in 900 mL 0.0033N hydrochloric | | | |
|---|---|---|---|---|
| | acid at 0 rpm | | | |
| | B1 (base alone) | | B2 (base + acid) | |
| Formulation | Paracetamol | Tramadol | Paracetamol | Tramadol |
| 120 sec | 1 | 1 | 78 | 86 |
| 180 sec | 1 | 1 | 83 | 93 |
| 300 sec | 2 | 3 | 85 | 96 |
| 15 min | 7 | 10 | 94 | 101 |
| 30 min | 13 | 25 | 101 | 103 |

| | | |
|---|---|---|
| Final pH | 2.8 | 2.5 |

Those skilled in the art will appreciate the invention described herein is susceptible to variations and modifications other than those specifically described. It is to be understood that the invention includes all such variations and modifications. The invention also includes the steps, features, compositions and compounds referred to or indicated in this specification, individually or collectively, and any and all combinations of any two or more of said steps or features.

1. A method for the rapid delivery of a therapeutic compound to the circulatory system of a patient comprising:
swallowing a swallow formulation whole and intact so that release of the therapeutic compound from the swallow formulation occurs downstream of the mouth in the gastrointestinal tract of the patient; and
wherein the swallow formulation comprises:
(a) the therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralize 0.01 to 9.0 millimoles of hydrochloric acid;
(c) wherein said carbonate is present in an amount (i) from about 1% to 50% by weight of the swallow formulation, and (ii) in a range that is greater than 0 mg carbonate, but less than an amount of carbonate at which the dissolution of the therapeutic compound from the formulation is unchanged or decreased from that achieved by a smaller amount of carbonate, when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm;
(d) wherein said range contains an amount of carbonate at which the maximum percentage of dissolution of said therapeutic compound occurs when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm; and
(e) wherein at least about 70% of the therapeutic compound is dissolved from the swallow formulation within 180 seconds, at 30 rpm when the dissolution is measured in United States Pharmacopoeia (USP) dissolution apparatus 2 with 900 mL 0.0033 N hydrochloric acid at 37° C.

2. The method of claim 1, wherein at least about 90% of the therapeutic compound is dissolved from the swallow formulation within 180 seconds at 30 rpm in USP dissolution apparatus 2 with 900 mL 0.0033 N hydrochloric acid at 37° C.

3. A method for the rapid delivery of a therapeutic compound to the circulatory system of a patient comprising:
swallowing a swallow formulation whole and intact so that release of the therapeutic compound from the swallow formulation occurs downstream of the mouth in the gastrointestinal tract of the patient; and
wherein the swallow formulation comprises:
(a) the therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralize 0.01 to 9.0 millimoles of hydrochloric acid and is present in an amount (i) from about 1% to 50% by weight of the swallow formulation, and (ii) in a range that is greater than 0 mg carbonate, but less than an amount of carbonate at which the dissolution of the therapeutic compound from the formulation is unchanged or decreased from that achieved by a smaller amount of carbonate, when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm;
(c) wherein said range contains an amount of carbonate at which the maximum percentage of dissolution of said therapeutic compound occurs when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm; and
(d) wherein at least about 5% of the therapeutic compound is dissolved from the swallow formulation within 300 seconds at 0 rpm when the dissolution is measured in United States Pharmacopoeia (USP) dissolution apparatus 2 with 900 mL 0.0033 N hydrochloric acid at 37° C.

4. The method of claim 3, wherein at least about 20% of the therapeutic compound is dissolved from the swallow formulation within 300 seconds at 0 rpm in USP dissolution apparatus 2 with 900 mL 0.0033 N hydrochloric acid at 37° C.

5. A method for the rapid delivery of a therapeutic compound to the circulatory system of a patient comprising:
swallowing a swallow formulation whole and intact so that release of the therapeutic compound from the swallow formulation occurs downstream of the mouth in the gastrointestinal tract of the patient; and
wherein the swallow formulation comprises:
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralize 0.01 to 9.0 millimoles of hydrochloric acid and is present in an amount (i) from about 1% to 50% by weight of the swallow formulation, and (ii) in a range that is greater than 0 mg carbonate, but less than an amount of carbonate at which the dissolution of the therapeutic compound from the formulation is unchanged or decreased from that achieved by a smaller amount of carbonate, when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm;
(c) wherein said range contains an amount of carbonate at which the maximum percentage of dissolution of said therapeutic compound occurs when the dissolution of the compound of the swallow formulation is measured at 90 seconds in United States Pharmacopoeia (USP) dissolution apparatus 2 with 900 mL 0.0033 N hydrochloric acid at 37° C. and at 30 rpm; and
(d) wherein
(i) at least about 70% of the therapeutic compound is dissolved from the swallow formulation within 180 seconds, at 30 rpm, and
(ii) at least about 5% of the therapeutic compound is dissolved from the swallow formulation within 300 seconds at 0 rpm when the dissolution is measured in United States Pharmacopoeia (USP) dissolution apparatus 2 with 900 mL 0.0033 N hydrochloric acid at 37° C.

6. The method according to any one of claim 1, 3 or 5, wherein the pH modulating agent of the swallow formulation comprises a base in the absence of an acidic pH modulating agent and the dissolution rate is greater than 5% at 300 seconds at 0 rpm.

7. The method of claim 5, wherein the dissolution rate is greater than 20% at 300 seconds at 0 rpm.

8. The method of claim 5, wherein the pH modulating agent of the swallow formulation comprises a base and an acid and the dissolution rate is greater than 5% at 300 seconds at 0 rpm.

9. The method of claim 8, wherein the dissolution rate is greater than 20% at 300 seconds at 0 rpm.

10. The method of any one of claim 1, 3 or 5, wherein the carbonate is selected from the group consisting of sodium carbonate, sodium bicarbonate, calcium carbonate, magnesium carbonate, ammonium carbonate, ammonium bicarbonate, potassium bicarbonate, sodium glycine carbonate, disodium glycine carbonate, arginine carbonate and lysine carbonate.

11. The method of any one of claim 1, 3 or 5, wherein the carbonate is water soluble.

12. The method of any one of claim 1, 3 or 5, wherein the carbonate is sodium carbonate.

13. The method of any one of claim 1, 3 or 5, wherein the carbonate is sodium bicarbonate.

14. The method of any one of claim 1, 3 or 5, wherein the carbonate is potassium bicarbonate.

15. The method of any one of claim 1, 3 or 5, wherein at least one of the pH modulating agents is a pharmaceutically acceptable acid.

16. The method of any one of claim 1, 3 or 5, wherein at least one of the pH modulating agents is a pharmaceutically acceptable acid selected from the group consisting of citric acid, tartaric acid, succinic acid, ascorbic acid, malic acid, fumaric acid, metatartaric acid, adipic acid, sodium acid citrate, potassium acid citrate, glycine citrate, potassium acid tartrate, sodium acid tartrate, aspartic acid, glutamic acid, glycine, leucine, tyrosine, tryptophan, glycine fumarate, glycine hydrochloride, monophosphate, glycine and combinations thereof.

17. The method of any one of claim 1, 3 or 5, wherein the swallow formulation further comprises a water uptake agent.

18. The method of any one of claim 1, 3 or 5, wherein the swallow formulation further comprises a water uptake agent wherein the water uptake agent is selected from the group consisting of cross-linked polyvinylpyrrolidone (crospovidone), croscarmellose sodium, sodium starch glycolate, starch, starch derivatives, hydroxypropylcellulose, low substituted hydroxypropylcellulose, hydroxypropylmethylcellulose, alginic acid, sodium alginate, calcium sulphate, calcium carboxymethylcellulose, microcrystalline cellulose, powdered cellulose, colloidal silicon dioxide, docusate sodium, guar gum, magnesium aluminum silicate, methylcellulose, polacrilin potassium, silicified microcrystalline cellulose, magnesium oxide, tragacanth, mannitol, sorbitol, xylitol, sucrose, lactose, fructose, maltose, polyethylene glycol, amino acids, cyclodextrin, urea and polyvinylpyrrolidone (povidone, PVP).

19. The method of any one of claim 1, 3 or 5, wherein the therapeutic compound is chosen from the group consisting of fexofenadine, pseudoephedrine, eletriptan, rizatriptan, metoclopramide, loperamide, codeine, diazepam, lorazepam, alprazolam, sildenafil, ondansetron, zolmitriptan, zolpidem, cetirizine, tramadol, a salt thereof and combinations thereof.

20. The method of any one of claim 1, 3 or 5, wherein the carbonate is present in an amount between 1% and 50% by weight of the swallow formulation.

21. The method of any one of claim 1, 3 or 5, wherein the swallow formulation comprises a pharmaceutically acceptable acid in an amount up to 50% by weight of the swallow formulation.

22. The method of any one of claim 1, 3 or 5, wherein the swallow formulation includes two or more therapeutic compounds chosen from the group consisting of basic drugs, amphoteric drugs, salts of basic drugs and salts of amphoteric drugs.

23. The method of any one of claim 1, 3 or 5, wherein the swallow formulation additionally includes a therapeutic compound chosen from the group consisting of acidic drugs, neutral drugs, salts of acidic drugs and salts of neutral drugs.

24. The method of any one of claim 1, 3 or 5, wherein said carbonate is present in an amount from about 1% to 27% by weight of the swallow formulation.

25. The method of claim 16, wherein the weight ratio of the uptake agent and the therapeutic compound in the swallow formulation is in the range of 3.5:1 to 223:1.

26. The method of any one of claim 1, 3 or 5, wherein said combination of carbonates and pharmaceutically acceptable acid is present in an amount from about 3% to 48% by weight of the swallow formulation.

27. A method for the rapid delivery of a therapeutic compound to the circulatory system of a patient comprising:
swallowing a swallow formulation whole and intact so that release of the therapeutic compound from the swallow formulation occurs downstream of the mouth in the gastrointestinal tract of the patient; and
wherein the swallow formulation comprises:
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralize 0.01 to 9.0 millimoles of hydrochloric acid;
(c) wherein said carbonate is present in an amount (i) from about 1% to 50% by weight of the swallow formulation;
(d) wherein at least about 70% of the therapeutic compound is dissolved from the swallow formulation within 180 seconds, at 30 rpm when the dissolution is measured in United States Pharmacopoeia (USP) dissolution apparatus 2 with 900 mL 0.0033 N hydrochloric acid at 37° C.

28. A method for the amelioration of the symptoms associated with a disease or disorder, including pain, fever, discomfort, migraine, nausea, insomnia, sleep disorders, allergic rhinitis, atopy and erectile dysfunction in a subject, the method comprising orally administering to a subject a swallow formulation wherein the swallow formulation is swallowed whole and intact so that release of the therapeutic compound from the swallow formulation occurs downstream of the mouth in the gastrointestinal tract; and
wherein the swallow formulation comprises:
(a) a therapeutic compound that is a base, a salt of a base, an amphoteric compound or a salt of an amphoteric compound, and
(b) an appropriate amount of one or more pH modulating agents wherein at least one pH modulating agent is a carbonate in an amount that will neutralize 0.01 to 9.0 millimoles of hydrochloric acid and is present in an amount from about 1% to 50% by weight of the swallow formulation,
wherein at least about 70% of the therapeutic compound is dissolved from the swallow formulation within 180 seconds, at 30 rpm when the dissolution is measured in United States Pharmacopoeia (USP) dissolution apparatus 2 with 900 mL 0.0033 N hydrochloric acid at 37° C.
